(19) United States
(12) Patent Application Publication
(10) Pub. No.: US 2019/0298213 A1
(43) Pub. Date: Oct. 3, 2019

(54) METHOD AND SYSTEM FOR PREDICTING HEART TISSUE ACTIVATION

(71) Applicant: King's College London, London (GB)

(72) Inventors: Steven Niederer, London (GB); Cesare Corrado, London (GB)

(73) Assignee: King's College London, London (GB)

(21) Appl. No.: 16/302,336

(22) PCT Filed: May 24, 2017
(86) PCT No.: PCT/GB2017/051461

§ 371 Date: Nov. 16, 2018

(30) Foreign Application Priority Data
May 25, 2016 (GB) 1609207.4

(51) Int. Cl. A61B 5/0452; A61B 5/042; A61B 5/046; G16H 50/50

(52) U.S. Cl. CPC A61B 5/04525; A61B 5/0422; A61B 5/046; A61N 1/362; A61B 2503/40; A61B 2562/0209; G16H 50/50

(57) ABSTRACT

Embodiments of the invention provide a method and apparatus that allow for a personalised heart tissue model to be generated, that models heart tissue electrophysiology behaviour at a personalised level, based upon activation measurements of an individual subject's heart in response to a number of predefined pacing protocols. The activation measurements are collected using a catheter placed onto the subject's heart, which is then paced via the catheter in accordance with the pacing protocols, and activation times of the heart tissue recorded. The activation measurements are used to generate a personalised tissue model, for example, by parameter matching the activation measurements with a large number of predefined sets of activation measurements, to determine the best-fit set; the best-fit set is then used as a personalised heart tissue model in a two or three-dimensional simulation of heart tissue activation in response to simulated stimulation.

### TECHNICAL FIELD

Embodiments of the present invention relate to a method and system for characterising the electrophysiological properties of heart tissue in a subject human or animal body, and also to methods and system that apply the characterisations in a simulation for predicting the location of heart tissue activation anomalies within the subject's heart tissue. The identified locations can then subsequently be the subject of preventative treatment, for example by way of ablation, or the like.

### BACKGROUND TO THE INVENTION AND PRIOR ART

Atrial fibrillation (AF) is a supra-ventricular tachyarrhythmia characterized by uncoordinated atrial activation with consequent deterioration of mechanical function.

AF is the most common arrhythmia, affecting almost 2.5 million people in the US, [1] and is associated with an increased incidence of cardiovascular disease, stroke and premature death.

AF is commonly treated by radio frequency catheter ablation in drug refractory patients, [2], [3], [4]. However, many patients require multiple procedures to achieve sinus rhythm [5]. No consensus regarding the mechanisms that sustain fibrillation in the atrium has been reached; however local tissue properties, identified by complex fractionated electrograms [6] and focal impulse and rotor activation patterns [7], [8], and a heterogeneous atrial substrate [9] have been proposed to play a role in the induction and maintenance of AF.

Biophysical modelling provides a formal framework that combines our understanding of atrial physiology, physical constraints and patient measurements to make quantitative predictions of patient response to treatment. The models characterize the local cellular ionic properties, conductivity and propagation of electrical activation across myocardial tissue. These models have provided insights into the fundamental mechanisms responsible for arrhythmias in the ventricles and atria, [10], and have the capacity to optimize treatment plans for an individual patient's pathology. However, current models have failed to capture individual variation in electrophysiological properties across the atria that are potentially crucial to representing the individual's pathology.

### SUMMARY OF THE INVENTION

Embodiments of the invention provide a method and apparatus that allow for a personalised heart tissue model to be generated, that models heart tissue activation behaviour at a personalised level, based upon activation measurements of an individual subject's heart in response to a number of predefined pacing protocols. The activation measurements are collected using a catheter inserted onto the subject's heart, which is then paced via the catheter in accordance with the pacing protocols, and activation times of the localised heart tissue recorded as electrocardiographic measurements. The electrocardiographic measurements are then used to generate a personalised tissue model of the local heart tissue that was activated, for example, by parameter matching the activation measurements with a large number of predefined sets of activation measurements, to determine the single or distribution of optimal parameter sets. The optimal parameter sets or set is then used as a personalised heart tissue model for the localised tissue that was activated in a two or three dimensional simulation of heart tissue activation in response to simulated stimulation. The simulated activations are then recorded as two or three dimensional images showing the simulated activation patterns across the localised heart tissue that was activated. Where the simulated images show aberrant activation patterns, for example rotor or spiral activation patterns then the locations at which such simulated aberrant activation patterns, for example rotor or spiral activation patterns, occur can be considered as candidate locations for a subsequent tissue ablation from a catheterisation or surgical procedure, to address atrial or ventricular arrhythmias in the subject, for example atrial fibrillation. In order to obtain measurements for a different local area of the heart, the catheter may then be repositioned onto the different local area, and the method performed again for that local area.

In view of the above, from one aspect there is provided a method, comprising: receiving heart tissue electrophysiology data pertaining to heart tissue electrophysiology properties that has been measured in response to one or more heart tissue pacing regimes applied to a localised region of a heart of a human or animal subject; generating a personalised heart tissue electrophysiology model personalised to the subject in dependence on the heart tissue electrophysiology data for the localised region; simulating heart tissue electrophysiology patterns across the one or more localised regions of heart tissue using the personalised heart tissue electrophysiology model; and outputting the simulation results.

In one embodiment the outputting comprises generating a two or three dimensional image map of the one or more localised regions of heart tissue, and plotting the simulated heart tissue electrophysiology patterns thereon. The generated two or three dimensional image map may then be displayed to a user on a display.

In some embodiments of the invention the simulation is performed to identify regions of heart tissue that exhibit a pathological abnormality. For example, the pathological abnormality may be the ability of the tissue to support abnormal heart tissue activation patterns that cause heart tissue fibrillation. Of particular concern are abnormal heart tissue activation patterns that include rotor or spiral activation patterns, as these can be indicative of activation patterns that induce or sustain atrial or ventricular fibrillation.

In one embodiment the generating step comprises: comparing the heart tissue electrophysiology data with a plurality of sets of pre-computed simulation data; and identifying a single or multiple sets of pre-computed simulation data from the plurality of sets that provides a single or distribution of best-fit matches to the measured heart tissue electrophysiology data according to one or more fitting criteria.

In this above, the measured heart tissue electrophysiology data usually comprises conduction velocity (CV) and effective refractory period (ERP) measurements, and the plurality of sets of pre-computed simulation data comprise respective simulated CV and ERP measurements. In such a case the identifying may often comprise: determining candidate sets of the plurality of sets that substantially match the measured ERP measurements and have a simulated CV within a threshold difference of the measured CV; and for the determined candidate sets, ranking the candidate sets in dependence on differences between the simulated CV and the measured CV; the best-fit match set or a distribution of matched sets of pre-computed simulation data being selected as the personalised heart tissue electrophysiology model from the highest ranked or combination of highly ranked candidate sets.

In one embodiment the simulating comprises: performing a 2D or 3D simulation using the personalised heart tissue electrophysiology model, the simulation being performed by initiating a simulated spiral wave with a simulated stimulation pacing protocol and calculating simulated tissue activations results across a simulated 2D or 3D region of heart tissue. With such an arrangement a 2D or 3D simulation across a region of tissue can be undertaken, and a corresponding 2D or 3D image map illustrating the results of the simulation generated for output and display to the user. In such a case the 2D or 3D image map shows to the viewer the regions of heart tissue in which anomalous activation patterns, as simulated, may occur. These regions are then candidate regions for treatment, for example by surgical or catheter ablation.

In many embodiments of the invention the heart tissue electrophysiology data comprises conduction velocity data and effective refractory period data. These data sets can be directly measured from, or found from, activation time measurements obtained from electrocardiographic measurements taken by a multi-electrode catheter stimulated by appropriate pacing signals.

In this respect, in one embodiment the heart tissue electrophysiology data is obtained using a multi-electrode catheter, the electrodes of which being spatially separated from one another, pacing signals being applied in use to one or two of the electrodes or from a remote secondary catheter and electrogram recordings and corresponding activation times being determined from the other of the electrodes in the multi-electrode catheter.

The pacing signals may for example comprise a plurality of sequences of pacing and test pulses, a sequence comprising a plurality of regularly timed pacing pulses followed by at least one irregularly timed test pulse.

In order to allow effective refractory period to be measured, the time between the irregularly timed test pulse and the preceding regularly timed pacing pulse may be reduced from sequence to sequence until such point that the test pulse follows the preceding pacing pulse so quickly that no tissue activation is obtained from the test pulse.

The above pacing and measurement techniques may be performed separately from the personalised model generation and subsequent simulation, and usually in advance to allow input data into the model generation and simulation process. Therefore, from another aspect the present invention also provides a method comprising applying pacing signals to a subject's heart tissue using a multi-electrode catheter attached to the heart tissue to be tested, the electrodes of the multi-electrode catheter being spatially separated from one another, the pacing signals being applied in use to one of the electrodes or electrode pairs of the catheter or to a remote secondary catheter, and measuring electrocardiographic responses of the heart tissue at the other of the electrodes of the multi-electrode catheter.

Within the above the pacing signals may comprise a plurality of sequences of pacing and test pulses, a sequence comprising a plurality of regularly timed pacing pulses followed by at least one irregularly timed test pulse. Moreover, the time between the irregularly timed test pulse and the preceding regularly timed pacing pulse may be reduced from sequence to sequence until such point that the test pulse follows the preceding pacing pulse so quickly that no tissue activation is obtained from the test pulse. With such a pacing regime than ERP can be found.

In the above the method preferably further comprises recording the heart tissue electrograms and storing these for output. This allows the electrogram data to be obtained from the subject in advance, and to then be processed, without the need for the subject to be present.

Further features of embodiments of the invention will be apparent from the appended claims.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 shows a decapolar catheter configuration and dimensions. Dimensions are expressed in mm. Bipolar electrodes are determined by pairs (e1, e2), (e3, e4), (e5, e6), (e7, e8) and (e9, e10). The pacing stimulus is applied to the central poles, (e5, e6), highlighted by the grey ellipse.

FIG. 2 shows an example of numerically computed trans-membrane potential (black line) and bipolar electrode output (grey line). Trans-membrane potential was evaluated as the mean of the trans-membrane potentials of the two poles constituting the electrode.

FIG. 3: Left: L2 error distribution (bars) and cumulative distribution (thick line) evaluated for 247 set of randomly chosen parameters. Right: L∞ error distribution (bars) and cumulative distribution (thick line) evaluated for 247 set of randomly chosen parameters.

FIG. 4: Error distribution bars and best error distribution obtained by choosing the nearest candidate parameter set for each of target parameter set. (lines) for each parameter. Bottom right: recurrence of the maximum error for each parameter.

FIG. 5: Error distribution and CDF for the L2 error evaluated a posteriori on effective refractory period and CV restitutions for s=[700, 400] ms

FIG. 6: Case restitutions

FIG. 7: Path of the first filament for case 2, 4 and 5. For cases 4 and 5 filaments are plotted until break-up occurred. Case 4 rotor breaks up after t˜3910 ms. Case 5 shows an unstable spiral wave that breaks up rapidly into multiple wavelets before terminating at t˜1200 ms. Grayscale represents the time and is expressed in ms.

FIG. 8: Example of s1_s2 pacing protocol. In this sequence, s1 is kept fixed while s2 is decremented of 20 ms.

FIG. 9: Path of the first filament for case 1 to 5. For cases 1, 4 and 5 filaments are plotted until break-up occurred. Case 1 and 4 rotors break up after t˜2400 ms and t˜3910 ms respectively. Case 5 shows an unstable spiral wave that breaks up rapidly into multiple wavelets before terminating at t˜1200 ms. Colour represents the time and is expressed in ms

FIG. 10 is a diagram of a pacing data collection apparatus of a first embodiment of the invention.

FIG. 11 is a diagram of a tissue model generation and simulation apparatus of the first embodiment.

FIG. 12 is a flow diagram illustrating the steps involved in collecting activation data.

FIG. 13 is a flow diagram illustrating the steps involved in generating a personalised tissue model from the collected activation data.

FIG. 14 is a flow diagram illustrating the steps involved in simulating activation patterns using the personalised tissue model.

FIG. 15 is a flow diagram illustrating the overall process performed by a first embodiment of the invention.

### DESCRIPTION OF THE EMBODIMENTS

An embodiment of the invention will now be described, followed by further more in depth discussion of the operation of embodiments of the invention, and the results that can be obtained thereby.

Embodiments of the invention are directed at providing a technique which measures properties of a localised region of a subject's heart around a catheter, and in particular the conduction velocity (CV) and effective refractory period (ERP) of the region, and from the measurements generates a personalised heart tissue activation model specific to the localised region of the subject's heart. As will be discussed later, this model generation is performed by parameter fitting the measured results to a database of pre-computed numerical simulations to identify the best fitting parameter set for the localised region. Once the personalised tissue activation data model has been obtained, it is then used to simulate activation patterns in the modelled local region of heart tissue, from which an activation pattern map image illustrating activation patterns in the region of the heart tissue can be generated. Areas of the heart within the simulated region which then exhibit fibrillation caused by, for example, rotor or spiral activation patterns can then be identified in the map image, which areas can then be considered as good candidate areas for a subsequent surgical ablation procedure. Further details of the overall operation of some embodiments of the invention are given in FIG. 15, from which it will be seen that some embodiments of the invention generally provide four stages of processing.

With reference to FIG. 15, in the first stage, at step 15.2, the personalised tissue activation data is collected using a test pacing regime. As described in more detail later, this involves surgically or through a catheterisation procedure placing a multipolar catheter and potential a secondary pacing catheter onto a region of the heart tissue, and then using the electrodes on the multipolar catheter or the secondary pacing catheter to pace the heart with particular test pacing regimes, described later. Electrocardiogram measurements are then taken from the electrodes of the multipolar catheter that are not being used to apply the pacing signals. The electrocardiograms are then stored, for later processing. The electrocardiograms are processed to determine when the localised heart tissue to the catheter electrodes activates in response to the paced electrical stimulations, and this activation data is then stored, for later processing. If further local regions of the heart are required to be tested, the catheter may be relocated, and the method then repeated for the new local region of the heart around the catheter in the new position. Once electrocardiogram data for all desired localised regions has been obtained the test subject is no longer required to be present, and the remainder of the method described herein can then be performed without the presence of the test subject.

Once the activation data using the test pacing regime has been obtained, at step 15.4 the embodiment described herein acts to generate a personalised tissue activation model for a particular set of measurements from a particular location from the personalised tissue activation data. As described in more detail later, this is performed by undertaking a parameter fitting to find an optimal distribution or best-fit of a number of pre-computed numerical simulations that best fits the observed and recorded activation data. The optimal parameter set or sets that is identified is then used as the characterising tissue activation model for the subject 1.

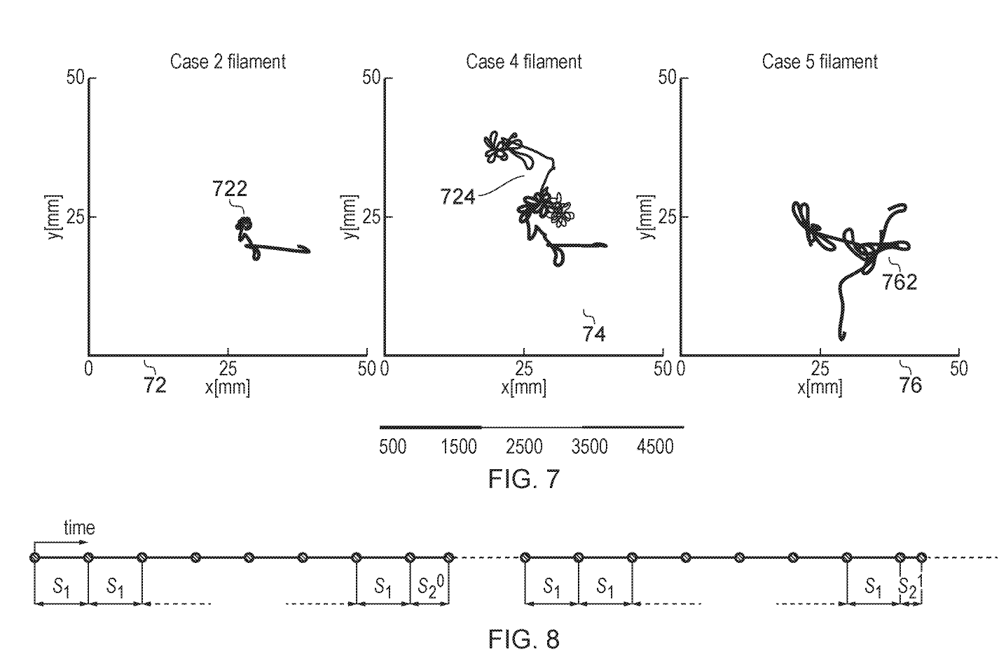

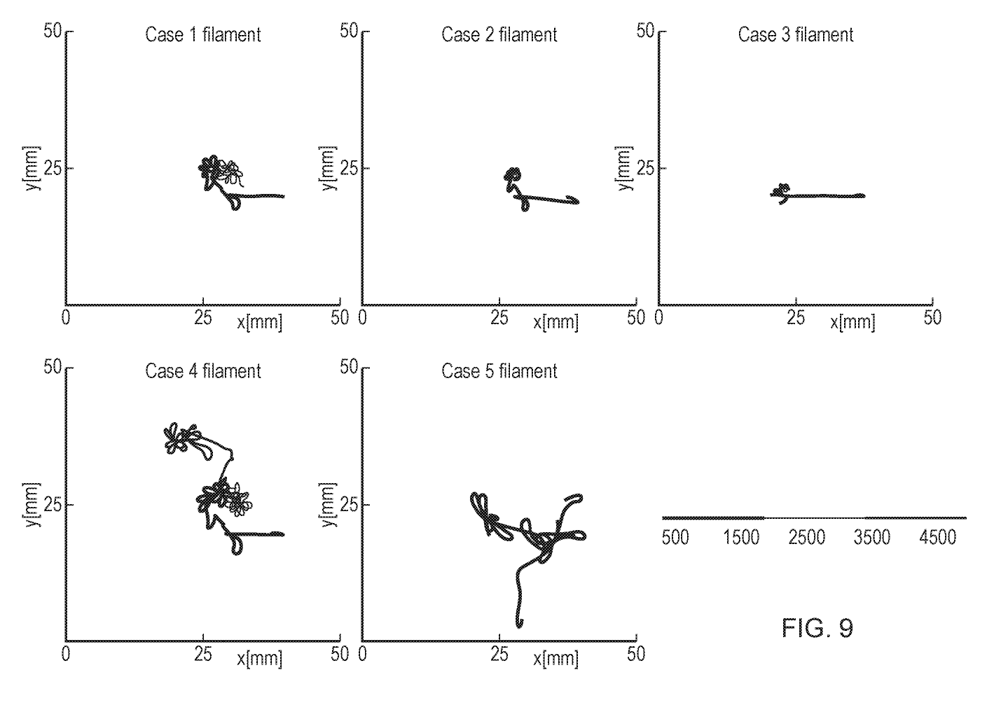

Having determined the personalised tissue activation model from the database of pre-computed numerical simulations at step 15.4, the identified personalised tissue activation model is then utilised at step 15.6 as the basis for a tissue characterisation simulation for the local region of the heart from which the measurements were taken, to determine whether the characterised tissue is capable of supporting re-entrant spiral activation patterns, as are often observed in atrial fibrillation. The result of the simulation, performed at step 15.6, is that a two or three dimensional map of heart tissue of the local region from which the measurements were taken, for example of part of the atrium, is generated which illustrates the activation pattern that is obtained from the simulation. Example activation pattern map images from such simulations are shown in FIG. 7 and FIG. 9, from which it can be seen that in some of the simulation cases demonstrated there are very well defined and volume limited spiral activation patterns that can be identified in the images. These results are significant, as the activation pattern map images provide an indication as to where spiral or rotor activation patterns may happen within the atrium, thus causing atrial fibrillation. The positions of the spiral or rotor activation patterns on the activation pattern map image can then form the basis of a subsequent surgical intervention, for example to ablate the atrial tissue at the identified positions, with a view to trying to stop the spiral pattern atrial fibrillation occurring.

In summary, therefore, embodiments of the invention described herein have four phases being the collection of electrocardiogram data for a localised region of the heart using the test pacing regime which is performed in the presence of the subject (s.15.2), the electrocardiogram data then being processed to determine tissue activation time data for the localised region (although this may also be performed later, outside the presence of the subject), and then subsequent phases being performed outside the presence of the subject, being those of generating a personalised tissue activation model for the localised region from the tissue activation data (s.15.4), and then using the personalised local tissue activation model to simulate activation patterns in the tissue (s.15.6), the simulation resulting in the generation of an activation pattern map for the localised region from which can be seen the location of spiral and rotor activation patterns (s.15.8). These spiral and rotor activation pattern positions are good candidates for subsequent surgical ablation treatment to attempt to address any atrial fibrillation occurring.

Having summarised the operation of embodiments of the invention, further details of the embodiments will now be described with respect to FIGS. 10 to 14.

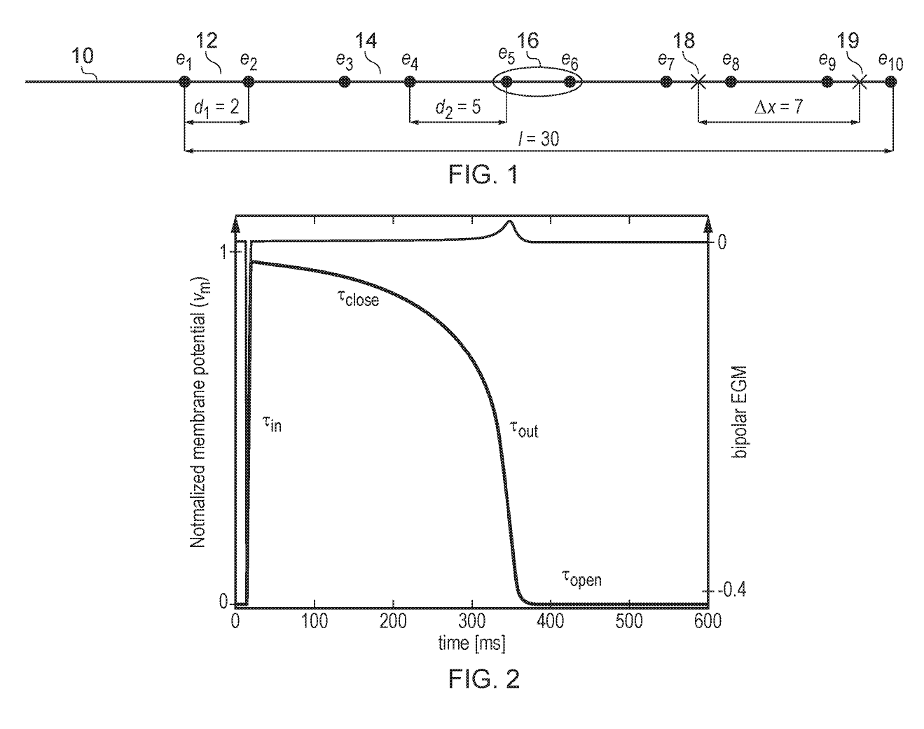

FIG. 10 illustrates an apparatus for collecting activation data from a subject 1. In this respect, a human or animal subject 1 is provided, having a heart 2 which is to be tested. A catheter 108 is inserted on the surface (which may be an inner surface or outer surface) of the atrium of the heart that is to be tested, for example, surgically. The catheter 108 is, in some embodiments, a decapolar catheter configuration, as shown in FIG. 1. Here five pairs of electrodes are provided along the catheter, with the central pair of electrodes 16 being the electrodes that receive pacing signals from a pacing and data recording apparatus 100. Pacing signals are provided to the central pair 16, and then bipolar recordings are taken from the other pairs of electrodes 12, 14, 18, and 19, as the pacing signals are conducted through the atrial heart tissue local to the catheter. The individual bipolar pairs of electrodes 12, 14, 18, and 19 are essentially recording, as electrocardiographic data, whether the local atrial tissue cells have been activated by the pacing signals applied at the central pair 16, and the relative times with respect to the application of the pacing signals to the central pair 16 that activation occurs. From the electrocardiographic data activation time measurements can then be calculated, and from the activation time measurements conduction velocity (CV) and effective refractory periods (ERP) of the local atrial tissue can be subsequently found.

In other embodiments, rather than the pacing signals being supplied to some of the electrodes of the same catheter which also contains the sensing electrodes, a second catheter having electrodes which receive the pacing signals may also be used, in which case the electrodes of the second catheter act to pace the heart, and the electrodes of the first catheter then act as sensing electrodes.

The pacing and activation data recording module 100 comprises an input and output interface 102 into which the decapolar catheter is connected, and which provides pacing signals to the decapolar catheter (or a second pacing catheter, if one is used), and records signals from the bipolar electrodes 12, 14, 18, and 19. The apparatus is further provided with a general purpose CPU 104, which is controlled by control program 1062 to control the apparatus 100 to operate as described herein. Additionally provided is pacing protocol data 1064, which controls the CPU 104 to cause the IO interface 102 to output pacing signals to the catheter 108 in accordance with a number of predetermined pacing protocols, such as those shown in FIG. 8, and tables S5 to S8, for example, described further below. Generally the pacing protocols comprise a number of regularly timed pacing signals, followed by a single irregularly timed signal with respect to the other signals, usually following at a shorter time period after the last regularly timed signal. The short time period is reduced further from set to set of pacing signals, with a view to determining the first shortened time period at which no tissue activation occurs, which then gives the ERP.

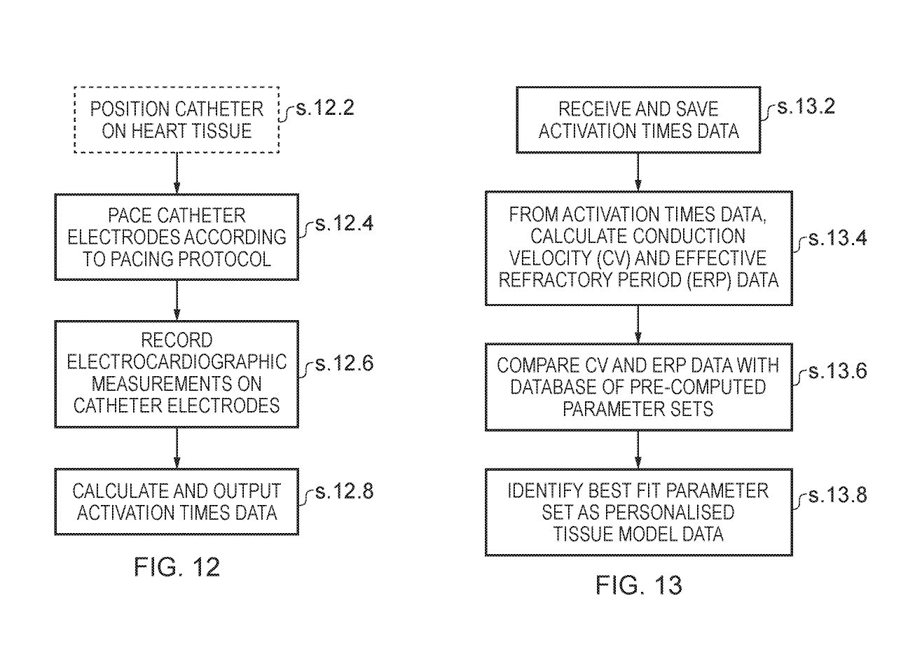

In operation, the pacing and activation data recording apparatus 100 operates in accordance with the process shown in FIG. 12. Here, after the decapolar catheter 108 has been positioned on the heart at step 12.2, for example in a surgical procedure, then once the catheter is in position and the subject 1 is ready, the control program 1062 controls the CPU 104 to cause the IO port 102 output pacing signals to the central electrodes 16 of the catheter 108 in accordance with the pacing protocols 1064. This step is performed at step 12.4. At the same time, electrocardiographic signals that then appear on the bipolar electrode pairs 12, 14, 18, and 19 in response to local atrial tissue activation in response to the pacing signals are then recorded via the IO port 102, as activation data 1066, on the computer readable storage medium (such as a hard disk, flash drive, or the like) 106. If a different local region of the heart is then required to be measured, the catheter may then be relocated on the heart tissue of the different local region, and the process repeated. Once all of the pacing protocols have been applied and the resulting activation data from the bipolar electrode pairs recorded for every local region desired to be measured, the presence of the subject 1 is no longer required, and the subject may be released, and the catheter 108 removed. Further details of the pacing protocols applied and the activation measurements thus obtained are given later, suffice to say here that the activation measurements comprise conduction velocity (CV) measurements, and effective refractory period (ERP) measurements, the CV and ERP measurements being found from activation time data that itself is obtained from the native electrocardiographic measurements taken by the electrodes on the catheter(s). For example, the CPU 104 may calculate the activation time data from the native electrocardiographic measurements taken from the catheter electrodes. As shown in FIG. 12, during the process, the electrocardiographic measurements from the bipolar electrode pairs 12, 14, 18, and 19 are recorded at step 12.6, and then the activation time data 1066, is calculated, and then subsequently output at step 12.8.

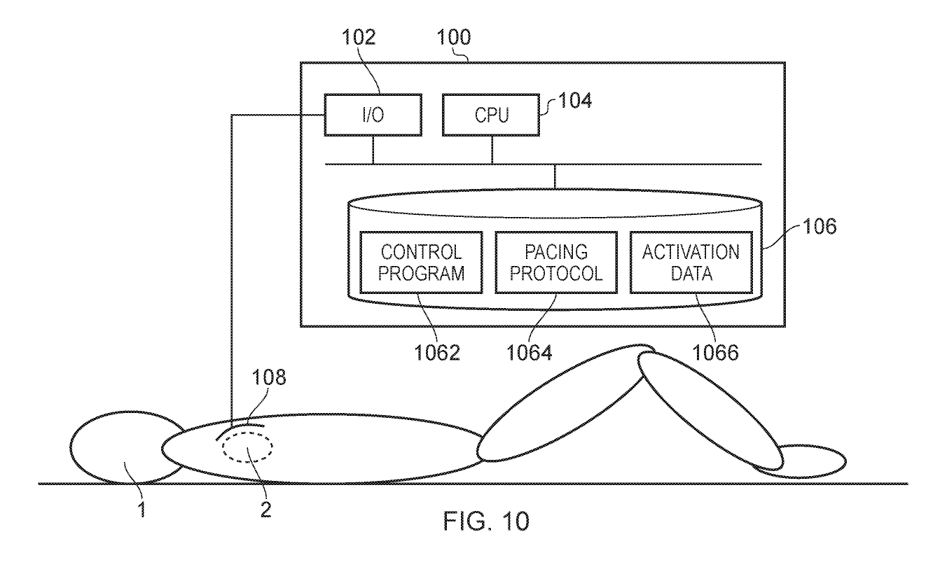

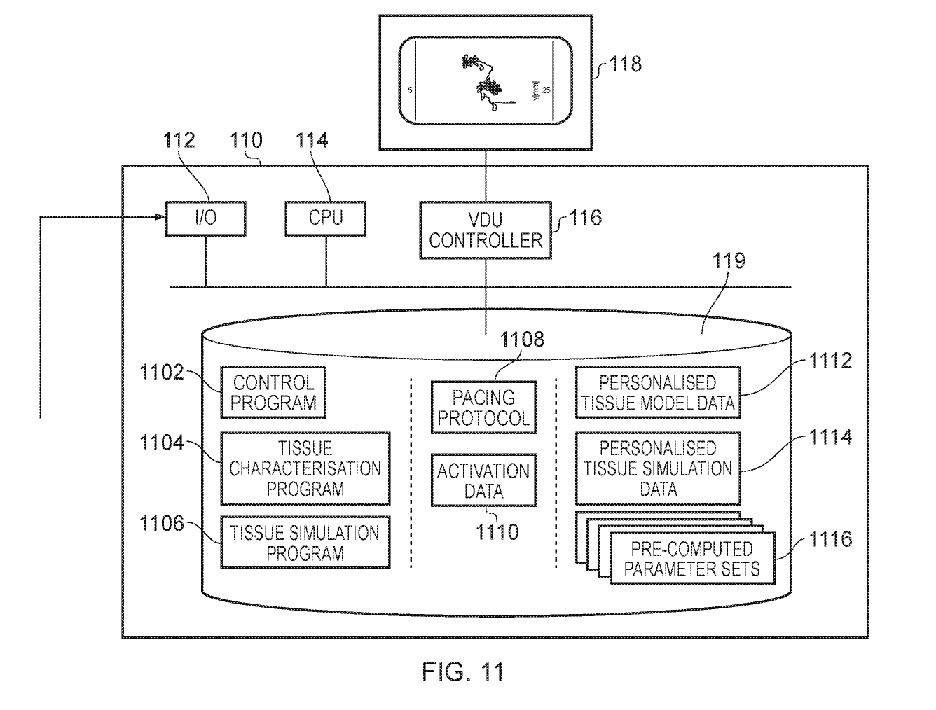

Once the activation data 1066 has been obtained, it may then be post-processed for each local region by an activation data processor unit 110, as shown in FIG. 11. The activation data processor unit 110 comprises a central processing unit 114, an input output port 112, and a visual display unit controller 116. The apparatus is further provided with a visual display 118, arranged to display any images of tissue activation maps that are generated.

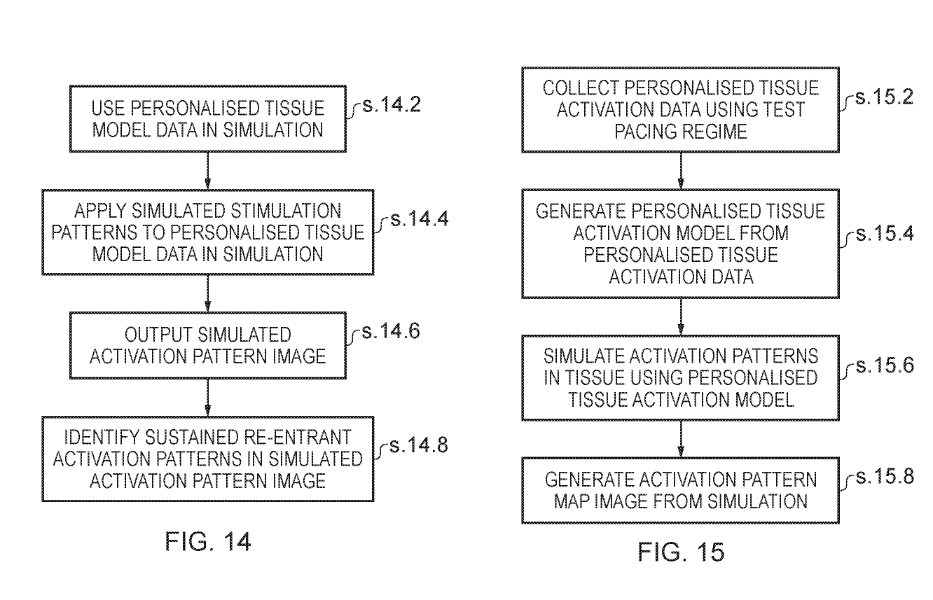

Also provided within the apparatus 110 is a computer readable storage medium 119, such as a hard disk, flash drive, or the like, on which is stored a control program 1102 which takes overall control of the operation of the apparatus, as well as a tissue characterisation program 1104, and a tissue simulation program 1106. The operation of the tissue characterisation program 1104 will be described later with respect to FIG. 13, and the tissue simulation program will be described with respect to FIG. 14. Also included on the computer readable storage medium 119 is the pacing protocol information 1108, which is the same pacing protocol information as was included in the apparatus 100 as pacing protocol information 1064. In addition, the activation data 1066 which is received from the apparatus 100 via the IO port 112 is stored as activation data 1110. As explained previously, the activation data 1110 comprises, for the pacing protocols, activation times as measured at the bipolar electrode pairs on the catheter, and from which CV and ERP can then be obtained.

Also stored on the computer readable storage medium 119, once generated, is personalised tissue model data 1112, which represents the personalised tissue model which is generated by the tissue characterisation program 1104 for a local region, from the activation data 1110 recorded for that region. In addition, once generated, there is also stored personalised tissue simulation data 1114 for each local region for which measurements were taken. This is generated by the tissue simulation program 1106, applying the personalised tissue model data 1112 to simulate atrial tissue electrophysiology across the respective local tissue volumes. The personalised tissue simulation data 1114 comprises, for each simulation performed, an output image map of heart tissue cell electrophysiology across the local atrial tissue for that simulation. Examples of the electrophysiology image maps that are obtained are shown in FIGS. 7 and 9, described further below. The generated tissue activation map images can be displayed by the VDU controller 116 on the VDU 118, as shown.

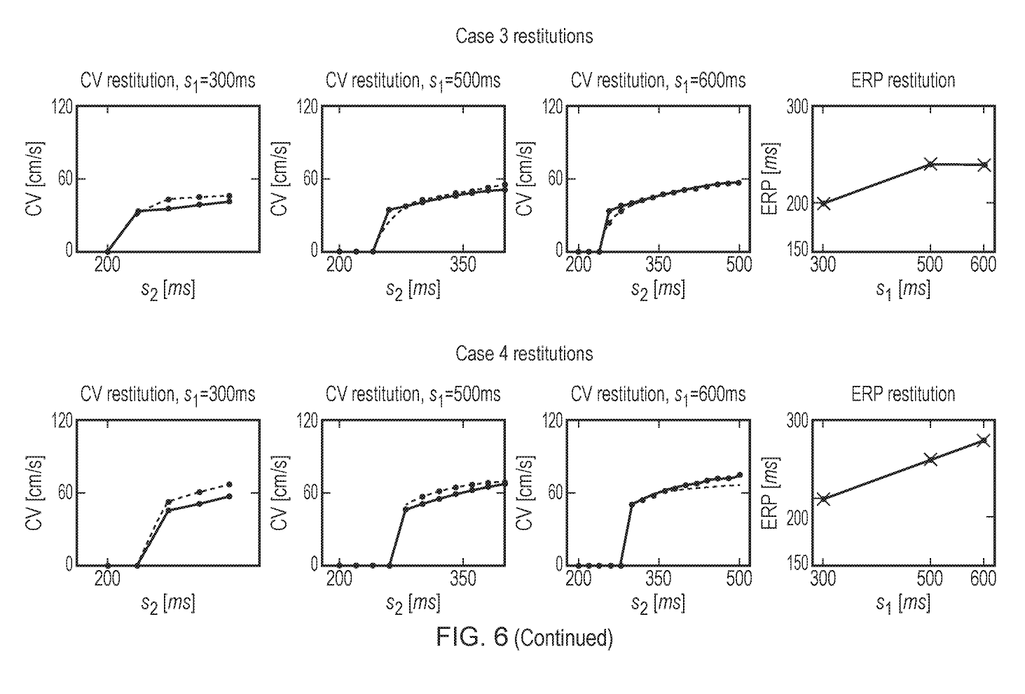

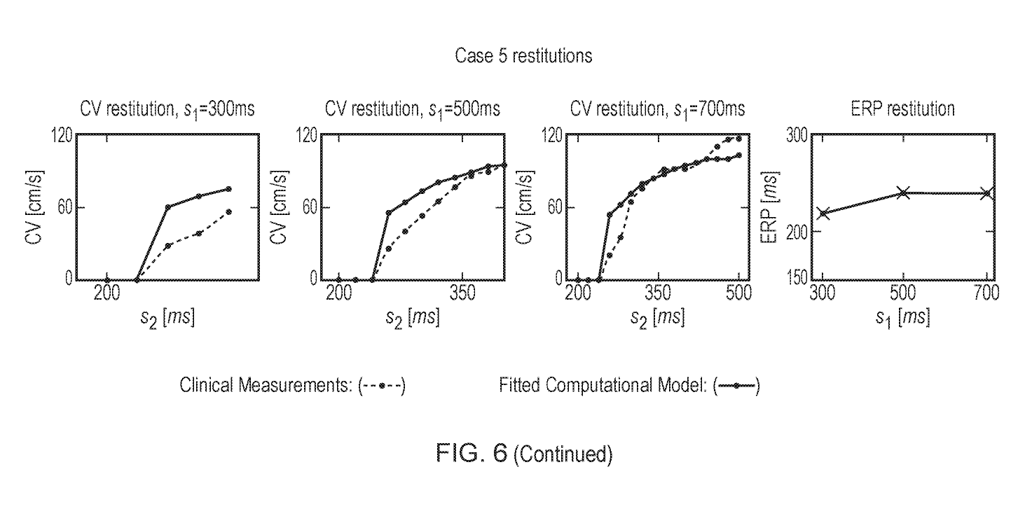

Also stored on the computer readable storage medium 119 are a number of pre-computed parameter sets 1116, forming a database of candidate simulation results for a large number of combinations of model parameters, as described in table 1 below, and shown in FIG. 6. The pre-computed parameter sets 1116 are used to compare the activation data 1110 thereagainst, to identify which pre-computed parameter set or distribution of parameter sets best matches the activation data. The best fit pre-computed parameter set or sets is then used as the personalised tissue model data 1112. Further details of the parameter fitting and matching process are given below. Turning now to FIGS. 13 and 14, the operation of the tissue characterisation program 1104 to generate the personalised tissue model data 1112 for a local region from the activation data 1110 from that region will now be described with respect to FIG. 13.

Firstly, when the activation data is received from the apparatus 100, at step 13.2, it is saved on the computer readable medium 119 as the activation data 1110. The activation data 1110 may be received at the apparatus 110 for example by being sent by email, or other file transfer or data transfer mechanism. Once received, the tissue characterisation program 1104 acts to calculate, from the activation times found from the electrocardiographic measurements, the conduction velocity (CV) across the atrial tissue, and the effective refractory period (ERP). The conduction velocity is the ratio between the bipolar electrode pair inter-electrode distance, and the time elapsed between the activation wave generated by a pacing signal propagating between the electrode pairs. The effective refractory period represents the smallest premature inter-pacing period where no conduction velocity is produced i.e. the smallest period between individual pacing signals which does not result in an activation signal propagating across the cells.

Once the conduction velocity and effective refractory period data has been obtained, then at step 13.6 the tissue characterisation program 1104 compares the data with a large number of pre-computed parameter sets, and performs a parameter best fit to determine which parameter set or distribution of parameter sets best fits the calculated observed data. Further details of the parameter fitting are given later. Once the best fit parameter set or sets have been identified, then that is recorded as the personalised tissue model data 1112, at step 13.8. The personalised tissue model data 1112 may then be used subsequently in a simulation by the tissue simulation program 1116 as described next.

FIG. 14 illustrates the operation of the tissue simulation program 1106. Firstly, at step 14.2 the personalised tissue model data is used to start a simulation of activation patterns in the local atrial tissue of a measured region. The simulation comprises applying simulated stimulation patterns to the personalised tissue model data and building up a simulated activation pattern from across the tissue of the local region. In this respect, the tissue is assumed to be homogenous across its width, as characterised by point measurements taken along the catheter location. The output of the simulation is an activation pattern image for the local region, as shown, for example, in FIGS. 7 and 9, which is then output at step 14.6. This image can then be saved on the computer readable storage medium 119, and displayed to the user on the visual display unit 118. Once displayed to the user, the user may then identify sustained re-entrant activation patterns in the simulated activation pattern image, to locate and identify those areas of the tissue where the re-entrant activation patterns occur. Alternatively, image processing may be performed on the image to identify the same patterns.

Because the image represents in 2D form the tissue of the atrium, the location of the activation patterns on the image corresponds to the location of the activation patterns within the tissue, and hence these locations can then become candidates for a subsequent surgical ablation procedure.

In some embodiments, a surgical or catheterisation ablation procedure can then be performed on the tissue corresponding to the location in the image, as guided by the simulated activation pattern image.

The above description therefore gives an overview of the operation of embodiments of the invention. Further details of the operation of the embodiment, and describing in further detail particular aspects thereof, are set out below, described with respect to the accompanying Figures.

As discussed above, characterizing the parameters of cellular ionic model across patient's atria is crucial for simulating the effects of tissue heterogeneity on AF. In this study we develop a clinically tractable protocol to quantify local tissue properties and encode them within a model of cellular electrophysiology. The study is separated into three sections. Firstly, we have quantified the accuracy of the model fitting protocol using in-silico data sets. Secondly, the fitting approach is applied to clinical measurements recorded from five cases. Finally, the potential clinical utility of this approach is demonstrated by using the fitted models to predict if characterized tissue regions can sustain a re-entrant spiral activation patterns and are potential ablation targets.

### II. METHODS

A. Computational Model

In this section we introduce the computational model adopted for simulating the propagation of the electrical stimulus across the atrial tissue. This model aims to numerically simulate catheter recordings to reproduce clinical electrograms.

Atrial tissue electrophysiology was modelled by the mono-domain simplification, [11], of the bi-domain electrophysiology model, [12], when intra- and extra-cellular conductivities are considered proportional up to a constant 1. To simulate recordings from a decapolar catheter a model of a 1D strip of atrial tissue was created. The decapolar catheter electrodes were placed along the tissue strip as shown in FIG. 1. The model was stimulated from electrodes (e₅, e₆) and bipolar recordings were calculated from the difference in extracellular potentials at electrode pairs (e₁, e₂), (e₃,e₄), (e₇,e₈) and (e₉,e₁₀). The distance (Dx) between pairs of electrodes for calculating conduction velocity corresponds to the distance between the baricentres of electrode pairs. Dimensions of the decapolar catheter are reported in FIG. 1.

The monodomain equations were discretized in space with a first order Finite Element Method (FEM) on a domain of length L=20 cm and with a discretization step of dx=200 mm. Time discretization of the partial differential equations were performed with the semi-implicit backward Euler method presented in [13], with a fixed time step of dt=0.1 ms. No mass lumping was applied. Electrograms were sampled at a rate of 5 kHz. Simulations were performed on the UK national super-computing facility ARCHER.

B. Choice of the Ionic Model

The ionic model was chosen to have the smallest number of parameters while capturing the measured conduction velocity (CV) and effective refractory period (ERP) restitution properties. Model complexity was selected to reflect the available clinical data. Physiological mechanisms, including cardiac memory and intracellular calcium handling were not recorded and so were not included in the model.

As clinical data were recorded using conventional catheters, only local activation time measurements are available; repolarisation times cannot be reliably recorded from conventional catheters on human atria. Thus, an s1_s2 protocol, as described in section II-C, will not provide a dynamic restitution curve for each s1, but it provides only an estimate of the steady state ERP for the s1 values evaluated. To estimate a dynamic ERP restitution curve would require the addition of a third stimulus that would need to be decremented, to estimate the ERP of each s2 pacing interval. This would drastically increase the duration of the protocol reducing our ability to use this approach to map multiple sites in the clinical setting. We thus adopt an ionic model that can be characterized by the available clinical measurements.

Two of the simplest models of cardiac physiology, that have a sharp upstroke, are the Aliev Panfilov (AP), [14] and Mitchell Schaeffer (MS), [15]. Each model has four ionic parameters and one diffusion coefficient parameter. The MS model was chosen as it is formulated in terms of depolarizing and repolarizing currents; however, the method developed here could equally be applied to the AP model. A complete description of the MS model and 1D fibre model are provided in the supplement. FIG. 2 shows a schematic of the simulated trans-membrane potential and the corresponding electrode signal. In the same Figure the parameter affecting each specific phase of the action potential (AP) is also shown.

C. Pacing Protocol and Activation Measurements

In clinical cases the atria were paced from the central poles (e₅,e₆) of the decapolar catheter placed on the roof of the left atrium (LA). Electrograms were measured from distal, (e₁,e₂), (e₉,e₁₀), and proximal, (e₃,e₄), (e₇,e₈) poles in a bi-polar configuration, with sampling frequency of 1 kHz (data set 1) and 4 kHz (data sets 2,3,4,5). A Biotronik UHS 300 device stimulator was used. A s1_s2 protocol was applied with s1=300, 500 and 600 ms, (data set 1-4) and s1=300, 500 and 700 ms (data set 5) and s2 values starting at 280, 400 and 500 ms, respectively. For each s1 pacing rate, s2 was decremented by 20 ms, down to the first s2 that did not capture, identifying the ERP. Before each premature s2 stimulus was applied, the tissue was pre-paced with 8 stimuli with a temporal interval of s1 to confirm reliable capture and achieve a steady state of activity. The chosen pacing protocol required <−5 min for its application.

For each bi-polar electrogram lead and for each inter-pacing interval the non-linear energy operator (NLEO), [16], was evaluated and filtered by a zero-phase window-based finite impulse response digital filter, [17] to eliminate oscillations. The time the electrical stimulus is applied and the stimulus duration are defined as t^{stim} and Dt^{stim} respectively. The activation time is defined as the time corresponding to the peak of the NLEO operator inside the time window [(t^{stim}+Dt^{stim}), (t^{stim}+Dt^{stim}+Dt^{act})]; peaks occurring outside the time window are considered anomaly and discarded. The choice of the interval Dt^{act} was based on manual calibration to minimize identification of early or delayed artefacts as the activation time, this yielded a minimum admissible CV of 14 cm/s. In this work Dt^{stim}=0.6 ms, while Dt^{act}=50 ms. A manual user check and validation on the activations was performed case by case.

By stimulating from the central poles of the decapolar catheter it is possible to record two sets of activation times at the same distance from the stimulus application site. This would provide two parameter sets at each catheter location. However, due to the curvature of the atria it is not possible to guarantee good contact between all electrodes and the atrial wall at each location. In none of the cases recorded here did we collect complete proximal and distal data sets. For this reason only one model is fitted to each catheter location.

D. Restitution Evaluation

In contrast to ventricular electrograms, [18], atria electrograms only display depolarization and do not show repolarization. Thus, from atrial electrograms it is possible to determine the activation time (depolarization) only. From activation times two restitution curves are directly available:
The CV restitution. For two adjacent electrode pairs, CV is evaluated as the ratio between the inter-electrode distance, (Dx) and the time elapsed between the activation wave, generated by the premature pacing (s2), propagating between the electrodes.
The ERP restitution. For each s1 inter-pacing interval, ERP represents the smallest s2 premature inter-pacing stimulus where no CV is produced.

ERP accuracy depends on the decrement step adopted for s2 (20 ms in this work); this accuracy allows us to constrain the model parameters while still ensuring a clinically compatible protocol duration.

E. Parameter Fitting

Model parameters were determined by comparing the clinically recorded or simulated CV and ERP restitution data with a data base of pre-computed numerical simulations to identify the best fitting parameter set. A data base of candidate simulation results for 99840 combinations of the model parameters summarized in Table I was created for the described pacing protocol.

TABLE I
Parameter values used for building the data set. A set of parameter
values ranging from the minimum to the maximum value in increments
of the step value is created. The data set of candidate solutions
was generated by models with each of the permutations of the Cartesian
products of all of the parameter value sets.

| | diffusion | | | | |
|---|---|---|---|---|---|
| | coefficient | tᵢₙ | tₒᵤₜ | tₒₚₑₙ | t_{close} |
| | (cm²/s) | (ms) | (ms) | (ms) | (ms) |
| min | 0.25 | 0.05 | 0.5 | 65 | 65 |
| max | 4.0 | 0.4 | 9.5 | 215 | 185 |
| step | 0.75 | 0.05 | 1.0 | 10 | 10 |

Clinical data used in this article always displayed 1:1 capture and had an ERP>200 ms. The MS model has been reported to exhibit pacemaker behaviour [19] where at the cellular scale, the MS model can spontaneously depolarize in the absence of a stimulus current, for some combinations of parameters. In the case of a tissue model, this appears as a focal activation, where a region of tissue spontaneously activates in the absence of a stimulus current or activation from a neighbouring cell. No evidence of this was found in the clinical data.

As a consequence, parameter sets with ERP<200 ms, that failed to yield 1:1 capture were excluded from the data set. To remove all parameter sets that exhibited pacemaker behaviour we applied three tests to each candidate parameter set. First the model was rapidly paced (3.33 Hz pacing) in a 0D model. Second, the parameter fitting pacing protocol was applied in a 1D model. Thirdly, a spiral wave was initiated in a 2D simulation (criterion for pacemaker activity are described in supplementary material).

Tests one and two were evaluated on all candidate parameters, giving a final data set of 51306 parameters sets. The third test is only performed on the fitted parameter sets to maintain computational tractability.

The parameter set that best fits clinical or simulated measurements is determined by the following two step algorithm:
The candidate ERP restitution curve and maximum CV value are compared against the corresponding curves for all the 51306 candidate parameter sets. A sub set of candidate parameter sets (I₁) is identified that matches the measured ERP restitution curve and have a maximum CV within 20% of the recorded value.
The L₂ norms of the difference between the measured CV restitution curves and the CV restitution curves for all candidate parameter sets in set I₁ are calculated and used to rank all candidate parameter sets.

Parameter sets that yield pacemaker behaviour in 2D simulations were identified and excluded as a final check in the parameter fitting algorithm, described above, to maintain computational tractability. This approach provided a robust and rapid fitting method taking approximately 6 minutes to find the optimal parameter set and ensure no pacemaker activity. The collective fitting performed here yields well constrained predictions, [20], [21], [22], even when individual parameters are poorly constrained.

### III. RESULTS

A. Validation with Synthetic Data

Error properties and robustness of our approach are evaluated by first generating a set of 247 models by randomly choosing parameter values within the [min, max] intervals reported in Table I. The test parameter set was created by generating a set of 1000 random parameter sets and then considering only combinations that provide a 1:1 capturing, an ERP>200 ms, at least one non zero CV value for s1=300 and s2=280, and did not exhibit pacemaker behaviour in 0, 1 or 2D simulations. The pacing protocol was then applied with s1=300, 500 and 600 ms and bipolar electrograms were numerically computed. A white noise with an intensity equal to 10% of the maximum absolute value of the electrode output was added to each electrode output; restitutions were then evaluated by applying the procedure described in section II-D.

For each of the 247 models, the parameter set determined from the fitting process was compared with the known true solution. All errors presented here are relative, reported as the percentage change between the known and the fitted value. The L₂ is the mean squared relative error on the 5 fitted parameters and furnishes a collective error estimate, where the contribution of each of the 5 parameters is taken into account. The L is the maximum relative error across the 5 fitted parameters and furnishes an error estimate based on the the parameter affected by the maximum error only.

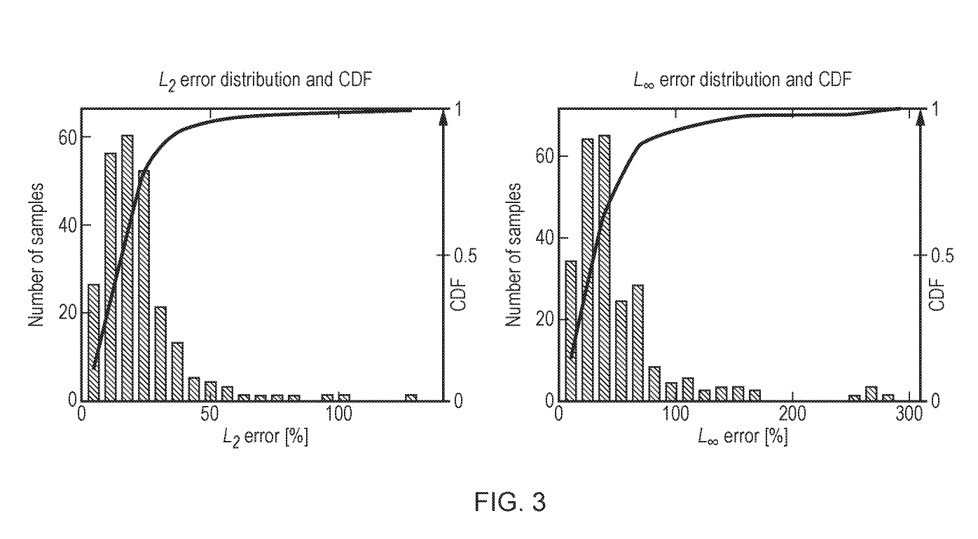

FIG. 3 shows the L₂ and L_{∞} error distributions and the corresponding cumulative distribution function (CDF). For the L₂ error, a mean error of 21.9% was found with a standard deviation of 16.1%. As depicted by the CDF, 95% of the estimated parameters analysed here have a L₂ error not greater than 40%. For the L error, a mean error of 48.1% was found and a standard deviation of 43.8%.

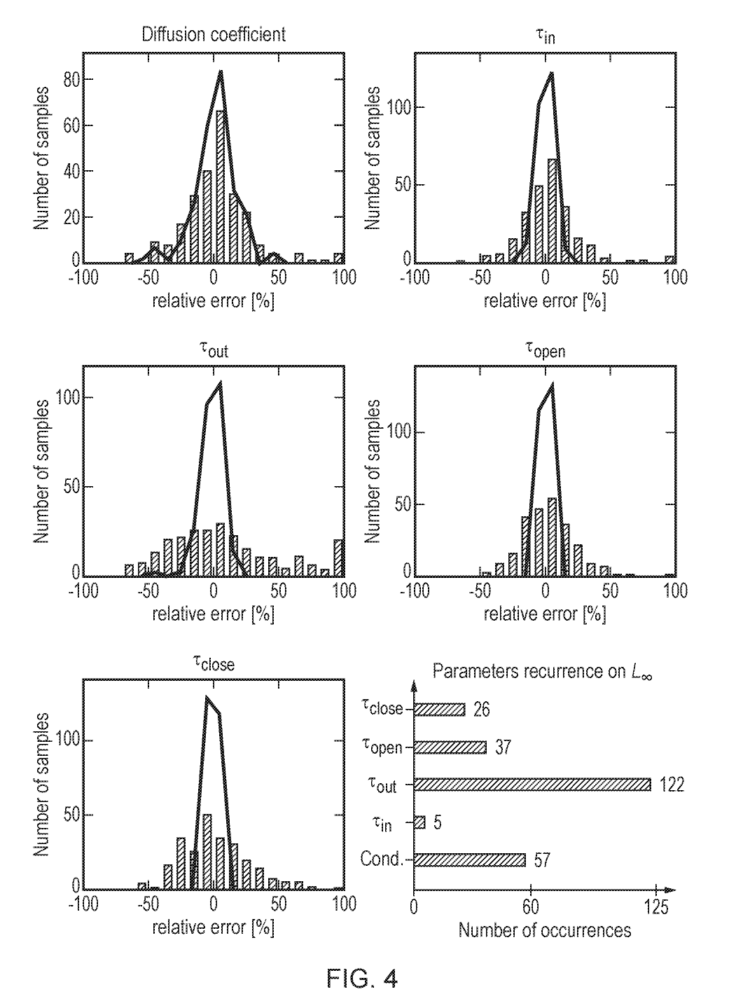

In FIG. 4 the signed relative error distribution is shown for each parameter, together with the relative difference between the selected parameter set and the optimal possible parameter set based on the nearest data base parameter set to the correct values. In the same Figure (bottom, right), the number of occurrences each parameter defines the L error is also reported. The best performances are obtained in estimating the diffusion coefficient (2.5±30.6%), tᵢₙ(3.6±25.2%), t_{close} (1.9±26.4%) and tₒₚₑₙ (2.2±22.1%) parameters. The parameter tₒᵤₜ (13.4±56.9%) is characterized by repolarization (FIG. 2) and is not well constrained by activation data.

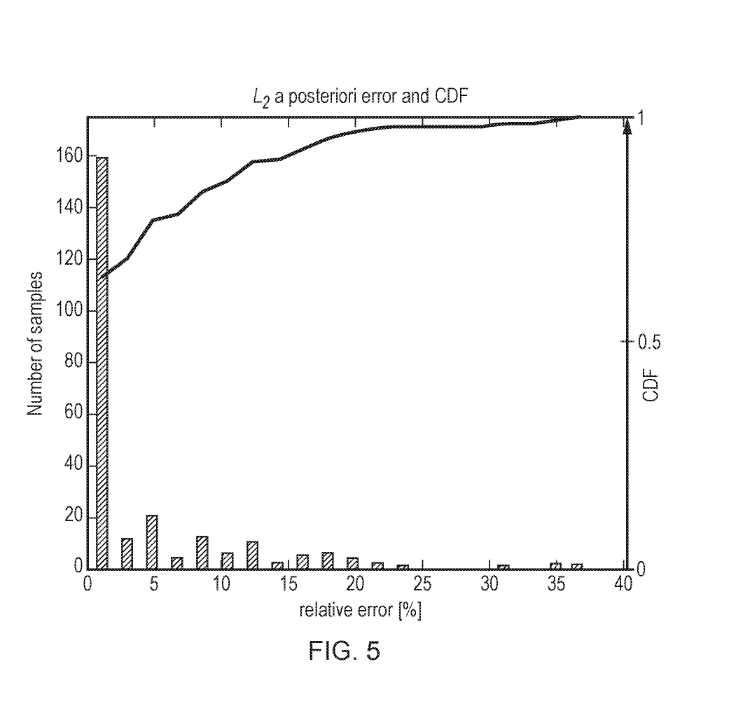

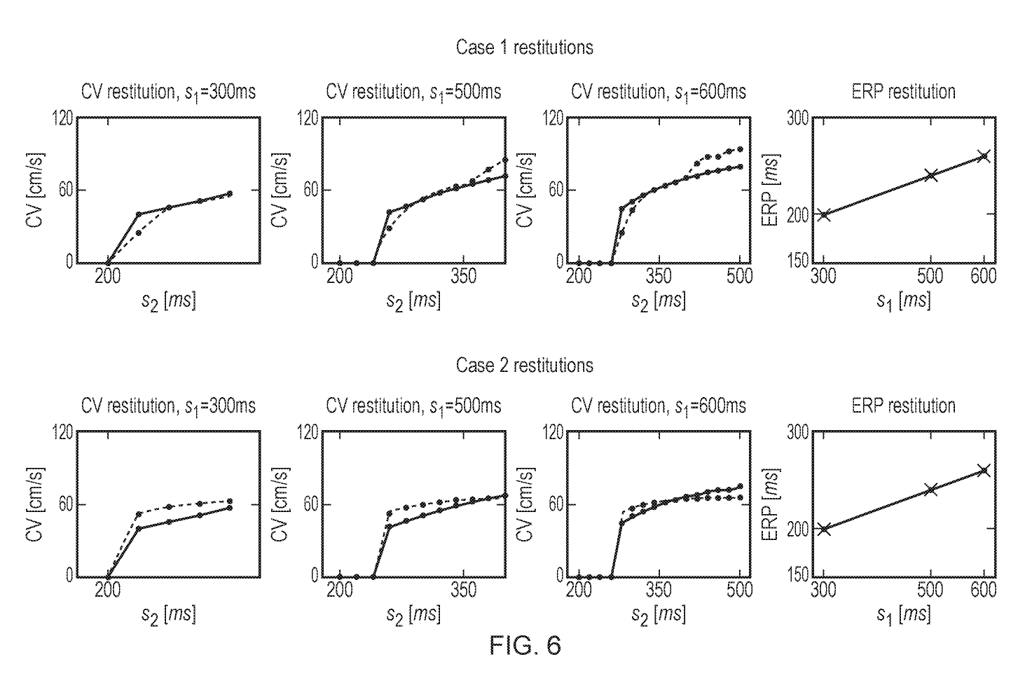

To test the impact of poorly constrained parameters on simulation results we compared the predicted CV and ERP restitution curves from the estimated parameter sets and the corresponding curves from the known parameter sets with an s1 pacing value of 400 and 700 ms that were not used in the original parameter fitting data. The L₂ relative errors with respect to ERP and CV restitution are evaluated for each of the 247 fitted parameter sets. FIG. 5 shows the distribution of error together with the CDF. The L₂ relative error was characterized by a mean value of 4.4% and a standard deviation of 6.9%. This confirms that, even though some parameters are not tightly constrained, the fitted parameter set gives a model that functionally represents the simulations generated with the known model parameters and is compatible with predicting functional tissue properties not used in the original fitting.

B. Application to Clinical Data

The model personalization protocol was then applied to 5 data sets recorded from patients suffering paroxysmal AF. All measurements used to constrain these models are provided in the supplementary material. ERP restitution (FIG. 6, panel 4) together with the maximum CV values were used to reduce the number of candidate parameter sets; CV restitutions (FIG. 6, panels 1-3) were then fitted to 30 measurements with different s1_s2 combinations the 3 curves generated for each s1 pacing rate. A unique parameter set was identified for each patient in 1 minute. Testing for 2D pacemaker behaviour took a further 5 minutes. Estimated model parameters for each data set are summarized in Table II below. Fitted ERP and CV restitution curves are shown in FIG. 6.

TABLE II
Estimated parameters for patients data sets

| | Case | Case | Case | Case | Case |
|---|---|---|---|---|---|
| Parameter | 1 | 2 | 3 | 4 | 5 |
| Diffusion | 1.0 | 1.0 | 1.0 | 1.4 | 1.4 |
| coefficient (cm²/s) | | | | | |
| tᵢₙ (ms) | 0.1 | 0.1 | 0.15 | 0.15 | 0.1 |
| tₒᵤₜ (ms) | 2.5 | 1.5 | 2.5 | 2.5 | 2.5 |
| tₒₚₑₙ (ms) | 165 | 135 | 205 | 115 | 95 |
| t_{close} (ms) | 115 | 155 | 115 | 145 | 115 |

To assess whether the accuracy of the parameter fitting would allow one to assess the regional differences in the electrophysiological properties of the atria, we compared the differences in the parameters fitted to clinical data within the context of the error estimated from the in silico study. The 95% confidence interval for the error of the fitted diffusion coefficient, tᵢₙ, tₒᵤₜ, tₒₚₑₙ, and t_{close}, from the in silico data is 40, 45, 100, 45 and 50%, respectively. In contrast, differences in parameter values in the clinical cases are up to 40, 50, 67, 116 and 35% for diffusion coefficient, tᵢₙ, tₒᵤₜ, tₒₚₑₙ, and t_{close}, respectively.

For the majority of parameters, the uncertainty in their estimated value is equal to or larger than the differences observed between clinical cases, reflecting the challenges in fitting parameters to sparse and noisy clinical data. The variation in the fitted tₒₚₑₙ values between clinical cases was larger than the uncertainty and could potentially be used to differentiate between tissue types.

C. Application Example: Tissue Characterization

Focal impulse and rotor mapping has recently been proposed as a novel ablation strategy [23], however this approach requires an expensive and large catheter and remains controversial. Encoding local tissue CV and ERP restitution properties within biophysical models provides a novel framework for predicting if local tissue properties are capable of supporting rotor or spiral activation patterns and may be useful in guiding ablation therapy. To predict if these tissue properties are compatible with supporting a re-entrant spiral activation pattern a spiral wave was initiated in a 2D simulation using a cross field stimulation pacing protocol with parameters from each of the 5 clinical cases. The original tissue properties were characterized by a point stimulus s1_s2 pacing protocol. While the model parameters were screened to remove pacemaker activity, where a small region of tissue spontaneously self activates, the models can support re-entrant spiral wave activation patterns where a wave of activation forms a self sustaining re-entrant circuit around the tissue (see movie 2). Models were simulated on 480 processors for 5000 ms taking 42 minutes of wall time. We observe three distinct spiral wave re-entrant activation patterns, as shown in FIG. 7: stable, meandering and breaking up. The only case to demonstrate spiral wave break up was Case 5, which had the largest discrepancy between modelled and measured CV restitution, particularly at high pacing frequencies (FIG. 6). The activation wave had a slower measured than modelled CV, which potentially allows a stable wave to exist in this tissue. Further cases would be required to confirm that we can reliably identify tissue that yields a predicted spiral wave break up. Rotor tip paths are shown for all cases in the supplement.

### IV. DISCUSSION AND PERSPECTIVES

In this work we present a robust and clinically feasible protocol and fitting algorithm for characterizing local atria tissue electrophysiology properties using a MS ionic model.

Previous studies have aimed to fit MS parameters in the ventricles to patient data [18],[24]. Relan et al. [18], used analytic derivations of action potential duration (APD) restitution curves and CV measurement to fit 3 of the 5 MS model parameters. Corrado et al. [24], estimated tᵢₙ and tₒᵤₜ, using a Kalman filter algorithm constrained by 12-lead ECG measurements. For parameters that were not fitted to patient data both studies used the default MS parameters, which were not derived from human data. In contrast, in this study all MS kinetic parameters were fitted to patient data. No error estimate was presented in these earlier studies so we cannot compare the relative accuracy of the different fitting approaches.

For the fitting method presented here, error quantification using synthetic data found that and that the available measurements still resulted in fitting errors of 13.4±56.9% for tₒᵤₜ, which is the least constrained parameter, while for t_{close} and tₒₚₑₙ error were (1.9±26.4%) and (2.2±22.1%) respectively. All three of these parameters are constrained by repolarization, which was measured with a coarse resolution in order to minimize the time taken to make recordings. Decreasing the s2 step size or the use of monophasic action potential catheters (MAP) [25], which provides a better measure of repolarization, may reduce the error in these parameters. However, reducing s2 limits the clinical feasibility of the described protocol, while MAP catheters will not give CV measurements unless combined with a multi-electrode catheter, which poses an increase in procedural complexity.

Despite the uncertainty in some of the parameters, we were able to demonstrate that the fitted parameters captured the functionality of the desired parameter set (see FIG. 5). This allows us to use this parameter personalization approach to generate local ionic models for patient specific simulations of atrial function.

The uncertainty in fitted model parameters limits the ability to use the proposed approach to differentiate between tissue types. The data-base fitting protocol was designed to be efficient and robust for clinical applications. Increasing the resolution of the database, holding uncertain parameters fixed or using the data base fit as an initialization for a non-linear optimization algorithm, such as Levenberg Marquardt, may improve the ability to differentiate between regional tissue types. However, the primary limitation is the weak sensitivity of the repolarization model parameters to the available clinical data. This is seen in FIG. 5, where the functionality is still captured, even in the presence of parameter uncertainties.

The measured maximum CV ranged between 60 and 100 cm/s (FIG. 6); these values are consistent with the values reported from clinical measurements [26]. Similarly, CV and ERP measurements are comparable with simulated restitutions generated from the more complex Courtermanche model [27]. The variation in CV measured at different atrial locations [26], highlights the importance of personalized atrial models that reflect the heterogeneity in electrophysiological behaviour seen throughout the atria.

The tᵢₙ, tₒᵤₜ and diffusion coefficient values were the most consistent across cases. Notably tᵢₙ and tₒᵤₜ were lower in all cases than in the original ventricular MS model (tᵢₙ=0.3, tₒᵤₜ=6.0), whereas the remaining fitted parameters spanned the original parameter values.

### V. LIMITATIONS

Clinical measurements are inherently noisy. By stimulating from the middle of the decapolar catheter we were able to record two potential data sets at each catheter location. However, due to noise and poor contact only the distal or proximal data sets were complete for any one location. The use of PentaRay catheters which provide five splines and 20 electrodes would result in higher density recordings and may improve the ability to characterize local electrophysiology properties.

Our model did not account for atrial curvature or wave-front curvature, both of which are likely to affect wave propagation. In the supplement we estimated the absence of wave curvature introduces an error of 6-10% on CV calculation. To incorporate these factors into a model would require two, or even three, dimensional models or bespoke models for each case, which would require a new database of model simulations for each measurement. This would significantly increase the computational burden of our approach. We could refine the data set discretization to reduce the error of the parameter fitting. However, given the accuracy of the measurements available the current data base discretization provides a reasonable balance between accuracy and over fitting and allows us to formally state the accuracy of the model parameter predictions.

Due to the limited number of patient cases it is not possible to conclude that the distinct tissue types identified in this study are relevant to the general AF population. Further studies involving larger numbers of cases would be required to confirm this result.

The model does not provide a complete description of known atrial myocyte physiology and does not account for cardiac memory, [28], calcium dynamics [29] or the effects of the parasympathetic nervous system [30]. The modelling philosophy adopted here is to choose the simplest model with the smallest number of parameters that can fit the available data. The MS model was able to replicate all of the clinical data collected providing no motivation to use a more complex and less well constrained model.

The MS model exhibits pacemaker behaviour in 0, 1 and 2D simulations that was not present in any of the clinical data sets recorded. The presence of pacemaker behaviour in the data base required the removal of a number of parameter sets. The stability of parameter sets was dependent on the dimensionality of the problem with some sets being stable in 0D and 1D simulations but unstable in 2D. This property has been reported previously [19] and is not unique to the MS model. Parameter sweeps of mouse, [31] and rabbit [32] biophysical ionic models also identify unviable parameter sets that fail to repolarize or that show a pacemaker behaviour. The introduction of a stability test addresses this issue. Pre calculating all 2D simulations is possible but comes at a high computational and data storage cost, so was not considered for this project but would reduce the parameter fitting process down to 1 minute.

### VI. FUTURE WORK

The proposed framework provides a pacing protocol and a method for rapidly and robustly creating models of local atrial tissue electrophysiology from clinical measurements. This approach has three potential clinical applications. Firstly, mapping the capacity of local tissue to support spiral waves using a readily available decapolar catheter may offer a novel alternative to identifying spiral waves. Secondly, combining our approach with measures of atria tissue fibrosis [33], wall thickness [34] or epicardial fat [35] may allow non-invasive indicators of pathological tissue types to be identified. Finally, developing maps of cellular properties across the atria allows for the creation of personalized models that capture both the patient atria anatomy but also an individual's heterogeneous tissue properties for guiding diagnosis, optimizing therapies and predicting outcomes.

The aim of this study is to develop, characterize and demonstrate a novel protocol for creating electrophysiological model of local tissue properties. The application of this technique to a selected cohort of patients will enable us to draw general physiological conclusions on atria electrophysiology.

### VII. CONCLUSIONS

In this work we presented a robust and clinically tractable protocol and fitting algorithm for characterizing local atrial electro-physiology properties by biophysical ionic cell models. We validated the novel method by means of synthetic data and demonstrated its clinical potential by applying it to 5 data sets recorded from paroxysmal AF patients undergoing pulmonary vein isolation.

### Supplementary Material

In this section we describe how we incorporated the Mitchell and Schaeffer ionic model (MS), [1] into the 1D fiber model of atrial tissue electrophysiology. We first introduce a bi-domain description [2] of the electrophysiology by characterizing the source term with the MS model, then we re-write the equations in terms of a dimensionless potential, we introduce the mono-domain simplification and we reconstruct the extra-cellular potential from a known trans-membrane potential.

The bi-domain model introduced in [2] is written as follows:

$$\begin{matrix}∇ \cdot ( {Σ}_{i} ∇ {φ}_{i} ) = {A}_{m} ( {C}_{m} \frac{\partial {V}_{m}}{\partial t} + {I}_{ion} ) & ( 1 ) \\ ∇ \cdot ( {Σ}_{e} ∇ {φ}_{e} ) = {A}_{m} ( {C}_{m} \frac{\partial {V}_{m}}{\partial t} + {I}_{ion} ) & ( 2 )\end{matrix}$$

where Vₘ, Φᵢ and Φₑ are the trans-membrane, intra-cellular and extra-cellular potentials respectively and are measured in mV, t is the time variable expressed in ms, Σ_{i,e} are the intra and extra cellular tissue conductivities and are expressed in S/cm, Aₘ is the cell surface per unit volume measured in $cm¹, Cₘ is the membrane capacitance expressed in μF/cm², Iᵢₒₙ is the ionic current measured in mA/cm². The right- and left-hand sides have units of mA/cm³ (volumetric source).

The MS model requires scaling the potentials so that the dimensionless voltage variable vₘ is between 0 and 1. Hence:

$${v}_{m} = \frac{{v}_{m} - {V}_{rest}}{{V}_{ap}} , {φ}_{i} = \frac{{φ}_{i} - {V}_{rest}}{{V}_{ap}} , {φ}_{d} = \frac{{φ}_{e} - {V}_{rest}}{{V}_{ap}} \qquad ( 3 )$$

where Vₐₚ is the magnitude of the action potential and Vᵣₑₛₜ is the resting value of the trans-membrane potential, in units mV. Substituting (3) into (1), (2) and dividing by Vₐₚ gives:

$$∇ \cdot ( {Σ}_{i} ∇ {φ}_{i} ) = {A}_{m} ( {C}_{m} \frac{\partial {v}_{m}}{\partial t} + \frac{{I}_{ion}}{{V}_{ap}} )$$

$$∇ \cdot ( {Σ}_{e} ∇ {φ}_{s} ) = {A}_{m} ( {C}_{m} \frac{\partial {v}_{m}}{\partial t} + \frac{{I}_{ion}}{{V}_{ap}} )$$

and dividing by AₘCₘ gives:

$$\begin{matrix}∇ \cdot ( {σ}_{i} ∇ {φ}_{i} ) = ( \frac{\partial {v}_{m}}{\partial t} + {J}_{ion} ) & ( 4 ) \\ ∇ \cdot ( {σ}_{e} ∇ {φ}_{s} ) = ( \frac{\partial {v}_{m}}{\partial t} + {J}_{ion} ) & ( 5 )\end{matrix}$$

where σ_{i,e}=Σ_{i,e}/(AₘCₘ) is a diffusion constant and has units of cm²/s and Jᵢₒₙ=Iᵢₒₙ/(CₘVₐₚ) is the scaled ionic current and has units of ms⁻¹. The right- and left-hand sides of eq (4) and (5) have units of ms⁻¹. Characterizing Jᵢₒₙ by MS, subtracting equation (5) by equation (4) yields equation (6), while summing equation (5) to equation (4) yields equation (7):

$$\begin{matrix}\frac{\partial {v}_{m}}{\partial t} = ∇ \cdot ( {σ}_{i} ∇ ( {v}_{m} + {φ}_{s} ) ) + \frac{{wv}_{m}^{2} ( 1 - {v}_{m} )}{{τ}_{in}} - \frac{{v}_{m}}{{τ}_{out}} + {J}_{stim} & ( 6 ) \\ ∇ \cdot ( {σ}_{i} ∇ ( {v}_{m} + {φ}_{e} ) ) + ∇ \cdot ( {σ}_{e} ∇ {φ}_{s} ) = 0 & ( 7 ) \\ \frac{\partial w}{\partial t} = \begin{matrix}\frac{1 - w}{{τ}_{open}} {v}_{m} \leq {v}_{cr} \\ \frac{- w}{{τ}_{close}} {v}_{m} > {v}_{cr}\end{matrix} & ( 8 )\end{matrix}$$

where Jₛₜᵢₘ represents an externally applied current and is expressed in ms⁻¹, v_{cr} represents a threshold activation potential, taken equal to 0.13 as from the original model, τᵢₙ, τₒᵤₜ, τₒₚₑₙ and τ_{close} are the 4 characteristic times of the 4 phases of the trans-membrane potential and are expressed in ms.

The mono-domain simplification [3] considers intra- and extra-cellular conductivities proportional up to a constant λ, such that:

σₑ=λσᵢ (9)

Introducing relation (9) into (7) and substituting into (6), it follows:

$$\begin{matrix}\frac{\partial {v}_{m}}{\partial t} = ∇ \cdot ( {σ}_{m} ∇ {v}_{m} ) + \frac{{wv}_{m}^{2} ( 1 - {v}_{m} )}{{τ}_{in}} - \frac{{v}_{m}}{{τ}_{out}} + {J}_{stim} & ( 10 ) \\ ∇ \cdot ( {σ}_{i} ∇ ( {v}_{m} + {φ}_{s} ) ) + ∇ \cdot ( {σ}_{s} ∇ {φ}_{s} ) = 0 & ( 11 ) \\ {σ}_{m} = \frac{{σ}_{i} {σ}_{e}}{{σ}_{i} + {σ}_{e}} = \frac{λ}{1 + λ} {σ}_{i} & \\ \frac{\partial w}{\partial t} = \begin{matrix}\frac{1 - w}{{τ}_{open}} {v}_{m} \leq {v}_{cr} \\ \frac{- w}{{τ}_{close}} {v}_{m} > {v}_{cr}\end{matrix} & ( 12 )\end{matrix}$$

where σᵢₙ represents the mono-domain equivalent diffusion coefficient. We note that equations (10) and (11) are now decoupled and it is possible to solve them independently.

In this work the tissue was modelled as a thin isotropic conductor. The effect of tissue micro-structure was not considered. Due to assumed local symmetry, negligible thickness [4] and the assumption that wave curvature has a secondary effect on conduction between recording bipoles (this latter hypothesis will be verified in section 3) the model was reduced to a 1D fibre model. From (11) and the assumption of no flux of intra and extracellular currents at the boundaries, the extra-cellular potential equilibrium equation is re-written as:

$${σ}_{i} \frac{{d}^{2} {v}_{m}}{{dx}^{2}} + ( {σ}_{i} + {σ}_{e} ) \frac{{d}^{2} {φ}_{s}}{{dx}^{2}} = 0 {σ}_{s} = {λσ}_{i} \\ \frac{d}{dx} ( {v}_{m} + ( 1 + λ ) {φ}_{e} ) = 0 \\ \frac{d}{dx} ( {v}_{m} + ( 1 + λ ) {φ}_{s} ) = 0 \qquad ( 13 )$$

The constant a on the right-hand side of (13) is fixed by imposing a zero spatial mean on the extra-cellular potential:

$$\int {v}_{m} = a$$

$${φ}_{s} = \frac{1}{( 1 + λ )} ( a - {v}_{m} )$$

2 Discretization of the MS Mono Domain Equations

To numerically solve the mono-domain Mitchell and Schaeffer equations (10) (12), we need to introduce a space discretization together with a time discretization. As far as space discretization is concerned, the trans-membrane potential Vₘ and the gate variable w were discretized with a first order Finite Element Method (FEM) on a domain of length L=20 cm by choosing a discretization step of dx=200 μm. The source term characterizing the ionic currents was treated with an ionic current interpolation [5]; no mass lumping was applied.

Time discretization was performed with a modification of the first order semi-implicit backward Euler method presented in [6]. A fixed time step dt=0.1 ms was chosen.

Denoting by Vₘⁿ, wⁿ the transmembrane potential and the gating variable at time tⁿ=n□t, the solution at time tⁿ⁺¹=tⁿ+dt is obtained as follows:
1. For each node, a 0D Mitchell and Schaeffer ionic model with initial conditions (vₘⁿ, wⁿ) is discretized by a Backward Euler method and solved implicitly via Newton iterations, leading to vₘ*, wⁿ⁺¹.
2. For each node, the source term of (10) is evaluated as:

$${J}_{ion} = \frac{{w ( {v}_{m}^{*} )}^{2} ( 1 - {v}_{m}^{*} )}{{τ}_{in}} - \frac{{v}_{m}^{*}}{{τ}_{out}} \qquad ( 14 )$$

3. The parabolic equation is solved with the ionic current determined in (14).

The choice of solving the parabolic diffusion equation with an implicit numerical scheme means we can avoid the restriction that the time step has to be of order dt−(dx²); moreover, the solution of the ionic model by an implicit scheme avoids the time step restrictions related to the stiff character of the depolarization wave. The interested reader can find more details in [7,8].

To ensure that we achieved a balance between numerical accuracy and speed given the large number of simulations performed we evaluated the error in the simulated conduction velocity (CV) introduced by out simulation time step. CV was calculated for each of the 5 parameter sets fitted to clinical data with the time step used in the data base dt=0.1 and again with dt=0.01. CV values and error are reported in Table S-I. The maximum error in CV was 2.6%. This corresponds to a difference in activation times of 0.15 ms. In context the electrograms can only be recorded at 4 kHz giving a 0.25 ms sampling interval.

3 Estimation of the Approximation Error Related to 1D Modelling

The approximation of the the atrial tissue with a 1D strip neglects the curvature of the propagation front and thus introduces a modelling error. To quantify the magnitude of the error arising from this approximation, we first perform 2D numerical simulations on a 5 cm×5 cm slab of tissue. The same decapolar catheter described in this work was adopted and the external stimulus is applied in the centre of the tissue slab, thus producing a circular propagating wave. Extracellular potentials are obtained, similarly to the 1D model as described above. From the simulated bipolar electrograms CV restitution curves were calculated. For each of the 5 parameter sets fitted to clinical data using the 1D model the CV restitution was calculated in the 2D model. The maximum absolute percentage CV error between the 1D and the 2D model for all s2 values for a given s1 value was used to quantify error introduced in the 1D model by ignoring potential curvature effects. The error ranges from 6.4-9.5% with results summarized in Table S-II.

4 a Criterion for Excluding Pacemaker Behaviour

The MS model demonstrates pacemaker behaviour, where a cell is activated in the absence of an external stimuli or diffusive currents, for specific combinations of parameter sets in 0D, 1D and 2D simulations. In 1D simulations, we test if the model is activated more times than it is stimulated to identify parameter sets that generate these ectopic beats. This is a rapid and low cost computation and is applied to all parameter sets in the data base. Any parameters sets exhibiting pacemaker behaviour are removed from the data base. Testing for pacemaker behaviour in 2D simulations is more computationally expensive and is only performed on parameter sets of interest. To test for pacemaker behaviour a spiral wave is initiated as described in the methods section and solved for 2500 ms on a coarser mesh (mean edge length of 235 m), with the transmembrane potential and gating variable sampled every 2 ms. For each solution the time derivative and Laplacian of the trans-membrane potential are evaluated as follows:

$$\frac{\partial {v}_{m}^{n + 1}}{\partial t} = \frac{{v}_{m}^{n + 1} - {v}_{m}^{n}}{dt}$$

$${∇}^{2} {v}_{m}^{n + 1} = \frac{1}{{σ}_{m}} ( \frac{\partial {v}_{m}^{n + 1}}{\partial t} - {J}_{in} ( {v}_{m}^{n + 1} , {w}^{n + 1} ) - {J}_{out} ( {v}_{m}^{n + 1} , {w}^{n + 1} ) )$$

For each time step we then evaluate the following conditions for all the mesh vertices:
The voltage is increasing, ∂vₘⁿ⁺¹/∂t>0
The depolarization is being driven by ionic and not diffusive currents,

$${∇}^{2} {v}_{m}^{n + 1} = \frac{1}{{σ}_{m}} {( \frac{\partial {v}_{m}^{n + 1}}{\partial t} - {J}_{in} ( {v}_{m}^{n + 1} , {w}^{n + 1} ) - {J}_{out} ( {v}_{m}^{n + 1} , {w}^{n + 1} ) )}_{< - M}$$

The cell is below the threshold value where ionic currents should not depolarize the cell, vₘⁿ⁺¹<v_{cr}

where M was chosen as 2% of the maximum amplitude of the Laplacian within the whole simulation; this criterion was adopted to account for possible numerical errors in the calculation of the Laplacian.

If these three criteria are satisfied for at least one point, we consider the model to be unstable.

5 Pacing Protocol Definition

In this section we describe the s1_s2 pacing protocol adopted for evaluating tissue restitution properties. The protocol is characterized by the pre-pacing value, s1, the initial value of the premature stimulus, s2⁰, and the decrement step for the premature stimulus, in this work taken equal to 20 ms. Once the s1 value is chosen, the tissue is pre-paced with 8 stimuli with a temporal interval of s1, followed by a pre-mature stimulus, s2. The sequence, depicted in FIG. 8 for two different values of s2, is repeated by decrementing the s2 value down to the first value not producing an action potential. The same procedure is then repeated by considering another couple of values s1, s2⁰; the values employed in this work are summarized in Table S-III for each case test.

6 Experimental Data Restitutions

In Tables S-IV-S-VIII CV and ERP restitutions for the 5 cases are reported. Note that, for each case and for each s1, values reported are truncated to the first s2 yielding an ERP 7 Spiral Wave Dynamics

In this section the predicted rotor tip path for the 5 cases is plotted in FIG. 9. The 5 cases demonstrate distinct spiral wave dynamics. Case 2 and 3 show a stable spiral wave, case 1 and 4 show a meandering spiral that break up after t˜2400 ms and t˜3910 ms, respectively. Case 5 shows an unstable spiral wave that breaks up rapidly into multiple wavelets before terminating at t˜1200 ms.

### TABLES

TABLE S-I
Evaluated conduction velocity for the 5 case sets for dt = 0.1 (left column)
and dt = 0.01 (central column) and relative error between the two different time
resolutions (right column). Each row correspond to each of the 3 pacing applied with
an inter-pacing interval of 600 ms

| | Case 1 | | | Case 2 | | | Case 3 | | | Case 4 | | | Case 5 | | |
|---|---|---|---|---|---|---|---|---|---|---|---|---|---|---|---|
| | | | (Err | | | Err | | | Err | | | Err | | | Err |
| | CV | | (%) | | | (%) | | | (%) | | | (%) | | | (%) |
| | dt = | | Err | CV | | Err | CV | | err | CV | | Err | CV | | Err |
| pacing | 0.1 | dt = 0.01 | (%) | dt = 0.1 | dt = 0.01 | (%) | dt = 0.1 | dt = 0.01 | (%) | dt = 0.1 | dt = 0.01 | (%) | dt = 0.1 | dt = 0.01 | (%) |
| 0 | 89.7 | 88.6 | 1.3 | 79.5 | 79.1 | 0.5 | 66.7 | 66.0 | 1.0 | 78.7 | 78.2 | 0.6 | 106.1 | 103.7 | 2.3 |
| 600 | 83.3 | 82.8 | 0.6 | 76.9 | 76.1 | 1.1 | 60.9 | 60.6 | 0.4 | 76.1 | 76.5 | −0.5 | 104.5 | 102.9 | 1.5 |
| 1200 | 84.3 | 82.8 | 1.8 | 76.9 | 76.5 | 0.5 | 61.4 | 60.9 | 0.9 | 76.1 | 76.5 | −0.5 | 104.5 | 102.9 | 1.5 |

TABLE S-II
Maximum relative error (%) on CV due to the
1D approximation compared to a 2D simulation

| | Case | Case | Case | Case | Case |
|---|---|---|---|---|---|
| s1 | 1 | 2 | 3 | 4 | 5 |
| 300 | 8.2% | 6.4% | 6.6% | 9.1% | 7.0% |
| 500 | 7.8% | 6.7% | 7.0% | 7.8% | 9.5% |
| 600 | 7.8% | 7.4% | 6.9% | 7.2% | 8.8% |
| (700) | | | | | |

TABLE S-III
Values of s1 and s2⁰ for characterizing the
adopted pacing protocol for each case test

| | | | | |
|---|---|---|---|---|
| s1 | 300 | 500 | 600 | 700 |
| [ms] | | | | |
| s2⁰ | 280 | 400 | 500 | 500 |
| [ms] | | | | |
| Case | 1, . . . , 5 | 1, . . . , 5 | 1, . . . , 4 | 5 |
| test | | | | |

TABLE S-IV
Case 1 restitutions

| s1 = 600 | | s1 = 500 | | s1 = 300 | | ERP | |
|---|---|---|---|---|---|---|---|
| 500 | 93.33 | 400 | 85.5 | 280 | 54.85 | 300 | 200 |
| 480 | 92.33 | 380 | 77.78 | 260 | 49.99 | 500 | 240 |
| 460 | 87.5 | 360 | 66.67 | 240 | 46.67 | 600 | 260 |
| 440 | 87.5 | 340 | 61.87 | 220 | 26.56 | | |
| 420 | 82.35 | 320 | 59.33 | 200 | 0 | | |
| 400 | 70 | 300 | 54.29 | | | | |
| 380 | 65.98 | 280 | 44.33 | | | | |
| 360 | 63.62 | 260 | 30 | | | | |
| 340 | 61.25 | 240 | 0 | | | | |
| 320 | 54.45 | | | | | | |
| 300 | 44.56 | | | | | | |
| 280 | 21.88 | | | | | | |
| 260 | 0 | | | | | | |

TABLE S-V
Case 2 restitutions

| s1 = 600 | | s1 = 500 | | s1 = 300 | | ERP | |
|---|---|---|---|---|---|---|---|
| 500 | 65.33 | 400 | 64.14 | 280 | 62.64 | 300 | 200 |
| 480 | 65.14 | 380 | 64.14 | 260 | 61.22 | 500 | 240 |
| 460 | 64.9 | 360 | 63.57 | 240 | 57.57 | 600 | 260 |
| 440 | 64.9 | 340 | 63.23 | 220 | 52.85 | | |
| 420 | 64.14 | 320 | 62.22 | 200 | 0 | | |
| 400 | 63.57 | 300 | 59.14 | | | | |
| 380 | 63.14 | 280 | 57.14 | | | | |
| 360 | 62.57 | 260 | 53.85 | | | | |
| 340 | 62.14 | 240 | 0 | | | | |
| 320 | 59.83 | | | | | | |
| 300 | 56.91 | | | | | | |
| 280 | 52.91 | | | | | | |
| 260 | 0 | | | | | | |

TABLE S-VI
Case 3 restitutions

| s1 = 600 | | s1 = 500 | | s1 = 300 | | ERP | |
|---|---|---|---|---|---|---|---|
| 500 | 56.33 | 400 | 55.57 | 280 | 46.75 | 300 | 200 |
| 480 | 56.33 | 380 | 52.83 | 260 | 45.89 | 500 | 240 |
| 460 | 56.14 | 360 | 50.9 | 240 | 43.9 | 600 | 240 |
| 440 | 53.83 | 340 | 47.46 | 220 | 32.44 | | |
| 420 | 51.91 | 320 | 45.83 | 200 | 0 | | |
| 400 | 50.28 | 300 | 41.42 | | | | |
| 380 | 48.33 | 280 | 38.18 | | | | |
| 360 | 46.67 | 260 | 24.54 | | | | |
| 340 | 44.44 | 240 | 0 | | | | |
| 320 | 41.18 | | | | | | |
| 300 | 37.78 | | | | | | |
| 280 | 34.15 | | | | | | |
| 260 | 26.41 | | | | | | |
| 240 | 0 | | | | | | |

TABLE S-VII
Case 4 restitutions

| s1 = 600 | | s1 = 500 | | s1 = 300 | | ERP | |
|---|---|---|---|---|---|---|---|
| 500 | 66.64 | 400 | 70.29 | 280 | 67.35 | 300 | 220 |
| 480 | 66.57 | 380 | 68.12 | 260 | 60.49 | 500 | 260 |
| 460 | 65.33 | 360 | 66.22 | 240 | 52.91 | 600 | 280 |
| 440 | 64.14 | 340 | 63.9 | 220 | 0 | | |
| 420 | 64.33 | 320 | 62.57 | | | | |
| 400 | 63.14 | 300 | 56.75 | | | | |
| 380 | 62.9 | 280 | 50.77 | | | | |
| 360 | 61.54 | 260 | 0 | | | | |
| 340 | 58.64 | | | | | | |
| 320 | 57.22 | | | | | | |
| 300 | 53.12 | | | | | | |
| 280 | 0 | | | | | | |

TABLE S-VIII
Case 5 restitutions

| s1 = 700 | | s1 = 500 | | s1 = 300 | | ERP | |
|---|---|---|---|---|---|---|---|
| 500 | 116.7 | 400 | 92.11 | 280 | 57.14 | 300 | 220 |
| 480 | 116.7 | 380 | 92.11 | 260 | 39.44 | 500 | 240 |
| 460 | 108.7 | 360 | 87.5 | 240 | 29.47 | 600 | 240 |
| 440 | 101 | 340 | 83.33 | 220 | 0 | | |
| 420 | 94.33 | 320 | 79.55 | | | | |
| 400 | 91.5 | 300 | 71.43 | | | | |
| 380 | 90.32 | 280 | 63.64 | | | | |
| 360 | 90.32 | 260 | 58.33 | | | | |
| 340 | 84.85 | 240 | 0 | | | | |
| 320 | 74.68 | | | | | | |
| 300 | 66.67 | | | | | | |
| 280 | 36.84 | | | | | | |
| 260 | 20.9 | | | | | | |
| 240 | 0 | | | | | | |

### REFERENCES

[1] S. Colilla et al., “Estimates of current and future incidence and prevalence of atrial fibrillation in the us adult population,” Am. J. Cardiol, vol. 112, no. 8, pp. 1142-1147, 2013.
[2] M. Haissaguerre et al., “Electrophysiological breakthroughs from the left atrium to the pulmonary veins,” Circ., vol. 102, no. 20, pp. 2463-2465, 2000.
[3] C. Pappone et al., “Atrial electroanatomic remodeling after circumferential radiofrequency pulmonary vein ablation: Efficacy of an anatomic approach in a large cohort of patients with atrial fibrillation,” Circ., vol. 104, no. 21, pp. 2539-2544, 2001.
[4] H. Oral et al., “Pulmonary vein isolation for paroxysmal and persistent atrial fibrillation,” Circ., vol. 105, no. 9, pp. 1077-1081, 2002.
[5] R. Cappato et al., “Worldwide survey on the methods, efficacy, and safety of catheter ablation for human atrial fibrillation,” Circ., vol. 111, no. 9, pp. 1100-1105, 2005.
[6] K. Nademanee et al., “A new approach for catheter ablation of atrial fibrillation: mapping of the electrophysiologic substrate,” J. Am. Coll. Cardiol., vol. 43, no. 11, pp. 2044-2053, 2004.
[7] V. Swarup et al., “Stability of rotors and focal sources for human atrial fibrillation: Focal impulse and rotor mapping (firm) of af sources and fibrillatory conduction,” J. Cardiovasc. Electrophysiol., vol. 25, no. 12, pp. 1284-1292, 2014.
[8] J. M. Miller et al., “Initial independent outcomes from focal impulse and rotor modulation ablation for atrial fibrillation: Multicenter firm registry,” J. Cardiovasc. Electrophysiol., vol. 25, no. 9, pp. 921-929, 2014.
[9] M. O'Neill et al., “The stepwise ablation approach for chronic atrial fibrillation-evidence for a cumulative effect,” J. Interv. Card. Electr., vol. 16, no. 3, pp. 153-167, 2006.
[10] P. Colli Franzone, L. Pavarino, and G. Savard, “Computational electrocardiology: mathematical and numerical modeling,” in Complex Systems in Biomedicine, A. Quarteroni, L. Formaggia, and A. Veneziani, Eds. Springer Milan, 2006, pp. 187-241.
[11] M. Potse et al., “A comparison of monodomain and bidomain reaction-diffusion models for action potential propagation in the human heart,” IEEE Trans. Biomed. Eng., vol. 53, no. 12, pp. 2425-2435, 2006.
[12] L. Tung, “A bi-domain model for describing ischemic myocardial D-C potentials,” Ph.D. dissertation, MIT, 1978.
[13] M. Ethier and Y. Bourgault, “Semi-implicit time-discretization schemes for the bidomain model,” SIAM J. Numer. Anal., vol. 46, no. 5, pp. 2443-2468, 2008.
[14] R. R. Aliev and A. V. Panfilov, “A simple two-variable model of cardiac excitation,” Chaos, Solitons & Fractals, vol. 7, no. 3, pp. 293-301, 1996.
[15] C. Mitchell and D. Schaeffer, “A two-current model for the dynamics of cardiac membrane,” Bull. Math. Bio., vol. 65, pp. 767-793, 2003.
[16] J. F. Kaiser, “On a simple algorithm to calculate the ‘energy’ of a signal,” in Acoustics, Speech, and Signal Processing, 1988. ICASSP-88., 1988 International Conference on, 1990, pp. 381-384.
[17] S. K. Mitra and Y. Kuo, Digital signal processing: a computer-based approach. McGraw-Hill New York, 2006, vol. 2.
[18] J. Relan et al., “Coupled personalization of cardiac electrophysiology models for prediction of ischaemic ventricular tachycardia,” Int. Focus, vol. 1, no. 3, pp. 396-407, 2011.
[19] M. Rioux and Y. Bourgault, “A predictive method allowing the use of a single ionic model in numerical cardiac electrophysiology,” ESAIM: Math. Model. Num. Anal., vol. 47, pp. 987-1016, 7 2013.
[20] R. N. Gutenkunst et al., “Universally sloppy parameter sensitivities in systems biology models,” PLoS comp. bio., vol. 3, no. 10, p. e189, 2007.
[21] J. J. Waterfall et al., “Sloppy-model universality class and the Vandermonde matrix,” Phys. rev. lett., vol. 97, no. 15, p. 150601, 2006.
[22] K. S. Brown and J. P. Sethna, “Statistical mechanical approaches to models with many poorly known parameters,” Phys. Rev. E, vol. 68, no. 2, p. 021904, 2003.
[23] S. M. Narayan et al., “Focal impulse and rotor modulation ablation of sustaining rotors abruptly terminates persistent atrial fibrillation to sinus rhythm with elimination on follow-up: A video case study,” Heart Rhythm, vol. 9, no. 9, pp. 1436-1439, 2012.
[24] C. Corrado, J.-F. Gerbeau, and P. Moireau, “Identification of weakly coupled multiphysics problems. application to the inverse problem of electrocardiography,” J. Comp. Phys., vol. 283, pp. 271-298, 2015.
[25] M. R. Franz, “Method and theory of monophasic action potential recording,” Prog. Cardiovasc. Dis., vol. 33, no. 6, pp. 347-368, 1991.
[26] F. M. Weber et al., “Conduction velocity restitution of the human atrium—an efficient measurement protocol for clinical electrophysiological studies,” IEEE Trans. Biomed. Eng., vol. 58, no. 9, pp. 2648-2655, 2011.
[27] M. Courtemanche, R. J. Ramirez, and S. Nattel, “Ionic mechanisms underlying human atrial action potential properties: insights from a mathematical model,” Am. J. Physiol. Heart Circ. Physiol., vol. 275, no. 1, pp. H301-H321, 1998.
[28] D. G. Schaeffer et al., “An ionically based mapping model with memory for cardiac restitution,” Bull. Math. Bio., vol. 69, no. 2, p. 459, 2007.
[29] C.-C. Chou et al., “Intracellular calcium dynamics and anisotropic reentry in isolated canine pulmonary veins and left atrium,” Circ., vol. 111, no. 22, pp. 2889-2897, 2005.
[30] J. S. Floras, “Sympathetic nervous system activation in human heart failure: Clinical implications of an updated model,” J. Am. Coll. Cardiol., vol. 54, no. 5, pp. 375-385, 2009.
[31] J. O. Vik et al., “Genotype-phenotype map characteristics of an in silico heart cell,” Front. Physiol., vol. 2, no. 106, 2011.
[32] O. J. Britton et al., “Experimentally calibrated population of models predicts and explains intersubject variability in cardiac cellular electrophysiology,” Proc. Natl. Acad. Sci., vol. 110, no. 23, pp. E2098-E2105, 2013.
[33] N. F. Marrouche et al., “Association of atrial tissue fibrosis identified by delayed enhancement mri and atrial fibrillation catheter ablation: The decaaf study,” JAMA, vol. 311, no. 5, pp. 498-506, 2014.
[34] M. Bishop et al., “Three-dimensional atrial wall thickness maps to inform catheter ablation procedures for atrial fibrillation,” Europace, p. euv073, 2015.
[35] S. Sarin et al., “Clinical significance of epicardial fat measured using cardiac multislice computed tomography,” Am. J. Cardiol, vol. 102, no. 6, pp. 767-771, 2008.

### REFERENCES FROM SUPPLEMENTARY MATERIAL

[1] C. Mitchell and D. Schaeffer, “A two-current model for the dynamics of cardiac membrane,” Bull. Math. Bio., vol. 65, pp. 767-793, 2003.
[2] L. Tung, “A bi-domain model for describing ischemic myocardial D-C potentials,” Ph.D. dissertation, MIT, 1978.
[3] M. Potse et al., “A comparison of monodomain and bidomain reactiondiffusion models for action potential propagation in the human heart,” IEEE Trans. Biomed. Eng., vol. 53, no. 12, pp. 2425-2435, 2006.
[4] M. Bishop et al., “Three-dimensional atrial wall thickness maps to inform catheter ablation procedures for atrial fibrillation,” Europace, p. euv073, 2015.
[5] P. Pathmanathan et al., “Computational modelling of cardiac electrophysiology: explanation of the variability of results from different numerical solvers,” International journal for numerical methods in biomedical engineering, vol. 28, no. 8, pp. 890-903, 2012.
[6] M. Ethier and Y. Bourgault, “Semi-implicit time-discretization schemes for the bidomain model,” SIAM J. Numer. Anal., vol. 46, no. 5, pp. 2443-2468, 2008.
[7] M. O. Bernabeu et al., “Chaste: incorporating a novel multi-scale spatial and temporal algorithm into a large-scale open source library,” Philosophical Transactions of the Royal Society of London A: Mathematical, Physical and Engineering Sciences, vol. 367, no. 1895, pp. 1907-1930, 2009.
[8] J. Whiteley, “An efficient numerical technique for the solution of the monodomain and bidomain equations,” IEEE Trans. Biomed. Eng., vol. 53, no. 11, pp. 2139-2147, 2006.

Various further modifications may be made to the above described embodiments whether by way of addition, deletion or substitution to provide further embodiments, any and all of which are intended to be encompassed by the appended claims.

1. A method, comprising:
receiving heart tissue electrophysiology data pertaining to heart tissue electrophysiology properties that have been measured on a localised region of a heart of a human or animal subject and in response to one or more heart tissue pacing regimes applied to the localised region;
generating a personalised heart tissue electrophysiology model personalised to the subject in dependence on the heart tissue electrophysiology data for the localised region;
simulating heart tissue electrophysiology patterns across the localised region of heart tissue using the personalised heart tissue electrophysiology model; and
outputting the simulation results;
wherein the generating comprises:
comparing the heart tissue electrophysiology data with a plurality of sets of pre-computed simulation data; and
identifying a set of pre-computed simulation data from the plurality of sets that best-fit matches the measured heart tissue electrophysiology data according to one or more fitting criteria.

2. A method according to claim 1, wherein the outputting comprises:
generating a two or three dimensional image map of the localised region of heart tissue;
plotting the simulated heart tissue electrophysiology patterns on the image map; and
displaying the generated two or three dimensional image map to a user on a display.

3. A method according to claim 1, and further comprising repeating the steps for a second localised region of the heart of the human or animal subject.

4. A method according to claim 1, wherein the simulation is performed to identify regions of heart tissue that exhibit a pathological abnormality.

5. A method according to claim 4, wherein the pathological abnormality is the ability of the tissue to support abnormal heart tissue activation patterns that cause heart tissue fibrillation.

6. A method according to claim 5, wherein the abnormal heart tissue activation patterns include rotor or spiral activation patterns.

7. (canceled)

8. A method according to claim 1, wherein the measured heart tissue electrophysiology data comprises conduction velocity (CV) and effective refractory period (ERP) measurements, and the plurality of sets of pre-computed simulation data comprise respective simulated CV and ERP measurements, and the identifying comprises:
determining candidate sets of the plurality of sets that substantially match the measured ERP measurements and have a simulated CV within a threshold difference of the measured CV; and
for the determined candidate sets, ranking the candidate sets in dependence on differences between the simulated CV and the measured CV;
the best-fit match set of pre-computed simulation data being selected as the personalised heart tissue electrophysiology model from the highest ranked candidate sets.

9. A method according to claim 1, wherein the simulating comprises:
performing a 2D or 3D simulation using the personalised heart tissue electrophysiology model, the simulation being performed by initiating a simulated spiral wave with a simulated stimulation pacing protocol and calculating simulated tissue electrophysiology results across a simulated 2D or 3D region of heart tissue corresponding to the localised region for which measurements were obtained.

10. A method according to claim 1, wherein the heart tissue electrophysiology data comprises conduction velocity data and effective refractory period data.

11. A method according to claim 1, wherein the heart tissue electrophysiology data is obtained using a multi-electrode catheter applied to the localised region, the electrodes of which being spatially separated from one another, pacing signals being applied in use to one of the electrodes in the multipolar catheter or in a secondary pacing catheter and activations times being determined from the other of the multipolar catheter electrodes.

12. A method according to claim 11, wherein the pacing signals comprise a plurality of sequences of pacing and test pulses, a sequence comprising a plurality of regularly timed pacing pulses followed by at least one irregularly timed test pulse.

13. A method according to claim 12, wherein the time between the irregularly timed test pulse and the preceding regularly timed pacing pulse is reduced from sequence to sequence until such point that the test pulse follows the preceding pacing pulse so quickly that no tissue activation is obtained from the test pulse.

14. A method, comprising:
applying pacing signals to a localised region of a subject's heart tissue using a multi-electrode catheter attached to the heart tissue to be tested, the electrodes of the multi-electrode catheter being spatially separated from one another, the pacing signals being applied in use to one of the electrodes or electrode pairs of the catheter or from a remote secondary pacing catheter, and
measuring electrocardiographic responses of the localised region of heart tissue at the other of the electrodes of the multi-electrode catheter.

15. A method according to claim 14, wherein the pacing signals comprise a plurality of sequences of pacing and test pulses, a sequence comprising a plurality of regularly timed pacing pulses followed by at least one irregularly timed test pulse.

16. A method according to claim 15, wherein the time between the irregularly timed test pulse and the preceding regularly timed pacing pulse is reduced from sequence to sequence until such point that the test pulse follows the preceding pacing pulse so quickly that no tissue activation is obtained from the test pulse.

17. A method according to claim 14, and further comprising recording the electrocardiographic measurements, and finding therefrom heart tissue electrophysiology data for the localised region, and providing the heart tissue electrophysiology data for output.

18. A method according to claim 17, wherein the heart tissue electrophysiology data comprises heart tissue activation and/or recovery timing data.

19. A method according to claim 14 and further comprising relocating the catheter onto a second localised region of heart tissue, and repeating the applying and measuring steps to obtain electrocardiographic responses of the second localised region.

20. An apparatus, comprising:
one or more processors; and
one or more computer readable storage media, storing instructions that when executed by the processor cause the processor to operate to:
receive heart tissue electrophysiology data pertaining to heart tissue electrophysiology properties that have been measured on a localised region of a heart of a human or animal subject and in response to one or more heart tissue pacing regimes applied to the localised region;
generate a personalised heart tissue electrophysiology model personalised to the subject in dependence on the heart tissue electrophysiology data for the localised region;
simulate heart tissue electrophysiology patterns across the localised region of heart tissue using the personalized heart tissue electrophysiology model; and
output the simulation results;
wherein the generating of the personalised heart tissue electrophysiology model personalised to the subject further comprises:
comparing the heart tissue electrophysiology data with a plurality of sets of pre-computed simulation data; and
identifying a set of pre-computed simulation data from the plurality of sets that best-fit matches the measured heart tissue electrophysiology data according to one or more fitting criteria.

21. (canceled)

22. An apparatus, comprising:
means for applying pacing signals to a subject's heart tissue using a multi-electrode catheter placed on the heart tissue to be tested or from a secondary remote pacing catheter, the electrodes of the multi-electrode catheter being spatially separated from one another, the pacing signals being applied in use to one of the electrodes or electrode pairs of the multi-electrode catheter or from a secondary remote pacing catheter, and
means for measuring electrocardiographic responses of the heart tissue at the other of the electrodes of the multi-electrode catheter.
